(19) United States
(12) Patent Application Publication
(10) Pub. No.: US 2026/0112491 A1
(43) Pub. Date: Apr. 23, 2026

(54) On-Device Machine-Learning Processing For Baby Care Devices

(71) Applicant: Woddle Baby, Inc., Alamo, CA (US)

(72) Inventor: Shaker Rawanbakhsh, Alamo, CA (US)

(21) Appl. No.: 19/362,259

(22) Filed: Oct. 17, 2025

Related U.S. Application Data
(60) Provisional application No. 63/708,884, filed on Oct. 18, 2024.

(51) Int. Cl. G16H 50/20; A47D 15/00; G06F 3/16; G16H 50/30

(52) U.S. Cl. CPC G16H 50/20; A47D 15/001; G06F 3/167; G16H 50/30

(57) ABSTRACT

A baby changing pad is configured for on-device processing of user commands. The baby changing pad includes at least one microphone, at least one speaker, and one or more processors. The at least one microphone captures an audio stream. The one or more processors identify a user command from the audio stream by extracting one or more acoustic features from the audio stream. The one or more processors then generate a response to the user command that is output by the at least one speaker.

### CROSS REFERENCE TO RELATED APPLICATIONS

This application claims the benefit of U.S. Provisional Patent Application Serial No. 63/708,884, filed October 18, 2024, the entire disclosure of which is hereby incorporated herein by reference.

### FIELD

This disclosure generally relates to baby care devices and, more specifically, to on-device machine-learning processing for baby care devices.

### BRIEF DESCRIPTION OF THE DRAWINGS

This disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 is a diagram of an example environment associated with baby care.

FIG. 2 is a block diagram of an example internal configuration of a computing device.

FIG. 3 is a block diagram of an example operating environment associated with a baby care device.

FIG. 4 is a data flow diagram of an example associated with on-device machine-learning processing for baby care devices.

FIG. 5 is a data flow diagram of another example associated with on-device machine-learning processing for baby care devices.

FIG. 6 is a flow diagram of an example process associated with on-device machine-learning processing for baby care devices.

FIG. 7 is a block diagram of an example of an audio processing pipeline of a baby care device.

FIG. 8 is a block diagram of another example of an audio processing pipeline of a baby care device.

FIG. 9 is a conceptual block diagram of an example associated with a hybrid processing environment for an audio stream associated with baby care.

FIG. 10 is a conceptual block diagram of another example associated with a hybrid processing environment for an audio stream associated with baby care.

FIG. 11 is a conceptual block diagram of another example associated with a hybrid processing environment for an audio stream associated with baby care.

FIG. 12 is a conceptual block diagram of another example associated with a hybrid processing environment for an audio stream associated with baby care.

FIG. 13 is a block diagram of an example of a machine-learning model associated with processing an audio stream captured at a baby care device.

FIG. 14 is a flowchart of an example of a technique for on-device machine-learning processing for baby care devices.

FIG. 15 is a flowchart of another example of a technique for on-device machine-learning processing for baby care devices.

### DETAILED DESCRIPTION

Conventional baby care devices, such as baby monitors or smart changing pads, often rely on cloud-based servers to process complex data, for instance, physiological measurements. This reliance on cloud computing presents several technical challenges. For example, the round-trip time to send data to a cloud server, process the data, and receive a response may introduce latency, which degrades the user experience for real-time interactions. Furthermore, transmitting data, such as audio from a nursery or a baby's health information, to an external server raises data privacy and security concerns for caregivers. The functionality of such devices may be also contingent on a stable internet connection, rendering them less reliable in environments with intermittent or unavailable network access.

The technical problem is compounded by the hardware constraints inherent in many consumer-grade baby care devices. These devices may be equipped with resource-constrained hardware, such as low-power microcontrollers, which may lack the computational power and memory to execute large, conventional machine-learning models. Running complex algorithms for tasks like audio processing, command recognition, or physiological risk assessment locally on such hardware can be computationally intensive and has traditionally been considered impractical.

Existing on-device processing solutions may be generic and not optimized for the acoustic and data environment of infant care. For example, standard voice recognition models may not be robust enough to function accurately amidst the specific types of background noise found in a nursery, such as a baby crying, white noise machines, or respiratory sounds. This lack of specialization can result in a high rate of false activations or missed commands, rendering the voice interface unreliable and frustrating for the user.

Implementations of this disclosure address problems such as these by providing a technological framework for on-device machine learning tailored for a baby care device. As used herein, the term “baby care device” may refer to an electronic apparatus designed to assist in the monitoring, care, or analysis of an infant's well-being. For example, a baby care device may include a smart changing pad, a baby monitor, a smart bassinet, a smart bottle, a wearable sensor for an infant, or other similar devices. In some implementations, a baby care device may include a network of two or more devices configured to measure, share, receive, and/or aggregate data associated with the baby, including physiological data. For example, a baby care device may include a thermometer, weight scale, blood oxygen monitor, heart rate monitor, or other device capable of measuring or tracking physiological parameters of a baby. In some implementations, the baby care device may be configured to receive and/or measure input and output data associated with milk or formula feedings, or solid food intake, and diaper changes. In some implementations, the baby care device may be configured to receive and/or measure data associated with infant sleep duration, quality, and/or sleep staging. In some implementations, a baby care device is a specialized computing device equipped with sensors to gather data, processors to analyze that data locally, and communication components to interact with users or other systems. The disclosed subject matter may facilitate near real-time, low-latency interpretation of various sensor inputs directly on a resource-constrained device, enhancing data privacy and reliability by minimizing reliance on external cloud servers. This may be achieved through a multi-stage process that systematically reduces and transforms complex sensor data into a compact, feature-rich format suitable for analysis by a lightweight, purpose-built neural network model capable of running efficiently on the device's local hardware.

Some implementations include an optimized audio processing pipeline and a specialized neural network model that may execute directly on a baby care device, facilitating low-latency, private, and reliable audio command recognition and physiological data analysis. The audio processing pipeline may be initiated when one or more audio sensors, such as microphones, capture audio signals and an analog-to-digital converter (ADC) generates a digital audio stream. In some implementations, this stream may be an interleaved digital audio stream received by a processor set via an Inter-IC Sound (I2S) bus. To reduce the computational burden, the processor set may first perform data reduction operations. These operations may include down-sampling the digital audio stream from a first sample rate (e.g., 32,000 Hz) to a second, lower sample rate (e.g., 16,000 Hz). Subsequently, the processor set may generate a single-channel audio stream from the down-sampled stream, for example, by using a resample filter, which further reduces the amount of data to be processed.

Following data reduction, the system may perform an efficient feature extraction process. The processor set may segment the single-channel audio stream into a plurality of overlapping audio frames. This may be performed using a sliding window, which is a mechanism for analyzing a continuous data stream in small, sequential segments. For example, a sliding window may define frames of approximately 40 milliseconds in duration with an overlap of approximately 10 milliseconds. In some implementations, the sliding window may be implemented using a circular buffer. As used herein, the term "circular buffer" may refer to a fixed-size data buffer in which new data overwrites the oldest data once the buffer is full, which is a memory-efficient technique that avoids data-copying operations. An example of a circular buffer is a 1024-byte block of memory where incoming audio samples are continually, periodically, or in response to a trigger event written, with a pointer indicating the start of the most recent data block for analysis.

From each audio frame, the processor set may extract a set of acoustic features. As used herein, the term "acoustic features" may refer to a numerical representation of one or more sound characteristics within an audio frame that is more compact and informative than the raw audio waveform. An example of acoustic features is a set of Mel Frequency Cepstrum Coefficients (MFCCs). In some implementations, other features, such as a Mel Spectrogram, may be used. The process of extracting MFCCs may involve an initialization phase (e.g., computing a Hanning window and a Fast Fourier Transform table) and then, for each frame, computing a power spectrum and applying a Discrete Cosine Transform (DCT) to generate the coefficients. These acoustic features may be assembled into a feature tensor, which is a multi-dimensional array of numerical data formatted for input into a machine learning model. For example, the feature tensor may have a shape of [1, 16, 96], representing a single batch of 96 time frames, each with 16 acoustic features.

The extracted acoustic features may be provided to a machine-learning model to facilitate an inference operation. The machine-learning model may be tailored for infant care, and may be a neural network model, which is a computational model inspired by the structure of the human brain, including interconnected layers of nodes or "neurons". Examples of neural network models that may be used include a Long Short-Term Memory (LSTM) model, a transformer model, or a deep neural network (DNN) model. This model may be architected with specific layers, such as a flatten layer, a general matrix multiplication (GEMM) layer, and a sigmoid layer, to efficiently process the feature tensor and produce an output (e.g., a tensor of shape [1, 1]) representing a probability. The inference operation may identify at least one of a wakeword, a user command, or a type of infant vocalization (e.g., a baby cry) within the captured audio. To further enhance on-device efficiency, particularly for LSTM models, the inference may be performed incrementally, processing the audio stream in small chunks while carrying forward a hidden state and cell state between chunks to maintain context without needing to store the entire audio clip in memory.

A specific aspect of this disclosure is a method for creating a specialized neural network model, a process referred to as the "Model Factory". This process begins by generating a first dataset of synthetic audio samples corresponding to a target phrase. An augmented training dataset is then created by combining these synthetic samples with a second dataset of noise samples. As used herein, "augmented training dataset" may refer to a collection of data used to train a machine learning model that has been artificially expanded by adding modified copies of existing data or newly created synthetic data. In this disclosure, the augmentation is specific in that the noise samples include infant-related acoustic data, such as baby cry audio, respiratory noise audio, or heart beating noise audio, to make the model accurate in its target environment. The neural network model is then trained using this augmented dataset, and the final trained model is provided for deployment in an audio recognition application, such as a wakeword detection application, on an edge device including an MCU. The model may be provided in a standard format like ONNX and compiled into embeddable C code for deployment.

Finally, the disclosure provides for a flexible hybrid architecture. While the system is optimized for on-device inference, it may determine, based on an inference operation, that an identified user command cannot be fulfilled using on-device resources alone (e.g., a complex, open-ended question). In response to this determination, the system may transmit data to a remote cloud backend to leverage more powerful computational resources, such as a large language model. In some implementations, the device may transmit the raw digital audio stream, the extracted acoustic features, or text generated by an on-device speech-to-text engine. The system then receives a response from the cloud backend for providing to the user, for instance, via a speaker.

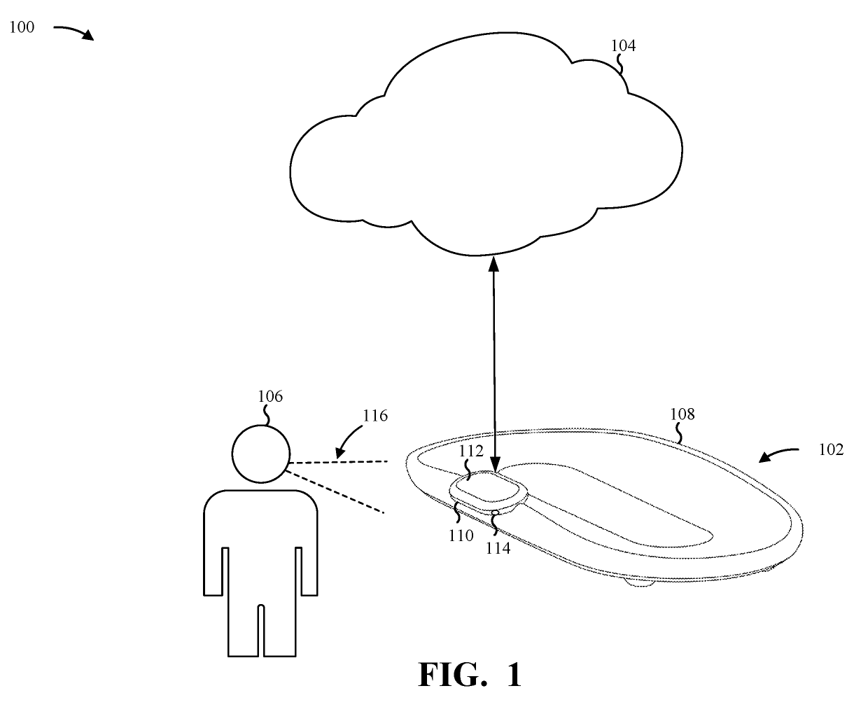

FIG. 1 is a diagram of an example environment 100 associated with baby care. The environment 100 includes a baby care device 102, a cloud system 104, and a user 106. The user 106 may interact with the baby care device 102, and the baby care device 102 may, in some implementations, communicate with the cloud system 104.

The baby care device 102 may be an electronic apparatus designed to assist in monitoring or caring for an infant. The baby care device 102 may be, be similar to, include, or be included in a smart changing pad, a baby monitor, a smart bassinet, or a wearable sensor. For example, the baby care device 102 may be configured to capture audio, process user commands, obtain physiological measurements, and provide responses or information to a user 106. In some implementations, the baby care device 102 is configured to perform on-device processing of data using one or more machine-learning models.

The cloud system 104 may be a remote computing environment that provides computational resources, data storage, and services accessible over a network. The cloud system 104 may be configured to communicate with the baby care device 102 to perform functions that supplement the on-device capabilities of the baby care device 102. For example, the cloud system 104 may host a large language model, a data store for training data, or a model engine for creating or refining machine-learning models. In some implementations, the baby care device 102 may transmit data, such as an audio stream or extracted acoustic features, to the cloud system 104 for processing and may receive a response or an updated machine-learning model from the cloud system 104.

The user 106 may be an individual, such as a parent or caregiver, who interacts with the baby care device 102. The user 106 may interact with the baby care device 102 through various modalities, for example, by providing voice commands, viewing information on a display, or using a companion application on a separate user device. For example, the user 106 may issue a voice 116 command to the baby care device 102 to inquire about an infant's status or to control a function of the baby care device 102.

As shown in FIG. 1, the baby care device 102 includes a changing pad 108, a control device 110, a display 112, and a microphone 114. In some implementations, two or more of the changing pad 108, the control device 110, the display 112, and the microphone 114 may be integrated into a single component. In some implementations, one or more of the changing pad 108, the control device 110, the display 112, and the microphone 114 may be implemented as separate, communicatively coupled devices. Although not shown in FIG. 1, the baby care device 102 may include other sensors, input components, output components, and communication components, such as an accelerometer, a light sensor, a proximity sensor, or a gyroscope. In some implementations, the baby care device 102 may include sensors for measuring physiologic parameters of an infant, such as temperature, heart rate, electroencephalogram activity, electromyogram activity, respiratory rate, blood oxygen saturation, or blood pressure. In some implementations, the baby care device 102 may include a speaker configured to deliver sounds, such as soothing music, or to provide outputs to the user 106, for instance, by playing synthesized speech or displaying alerts or prompts to the user 106. The baby care device 102 may further include a power source, such as a rechargeable battery. In some implementations, the baby care device 102 may include a wireless communication component for communication with external devices and systems, such as the cloud system 104. For example, the baby care device 102 may be configured to transmit digital data, such as a digital audio stream, extracted acoustic features, or a text string representing a user command to the cloud system 104.

The changing pad 108 may be a surface designed for changing an infant's diaper. The changing pad 108 may be integrated with one or more sensors to gather data about the infant. For example, the changing pad 108 may include physiological sensors configured to obtain physiological measurements of a baby, such as weight, temperature, or heart rate. In some implementations, the changing pad 108 is part of the baby care device 102 and provides a physical interface for the infant during care routines.

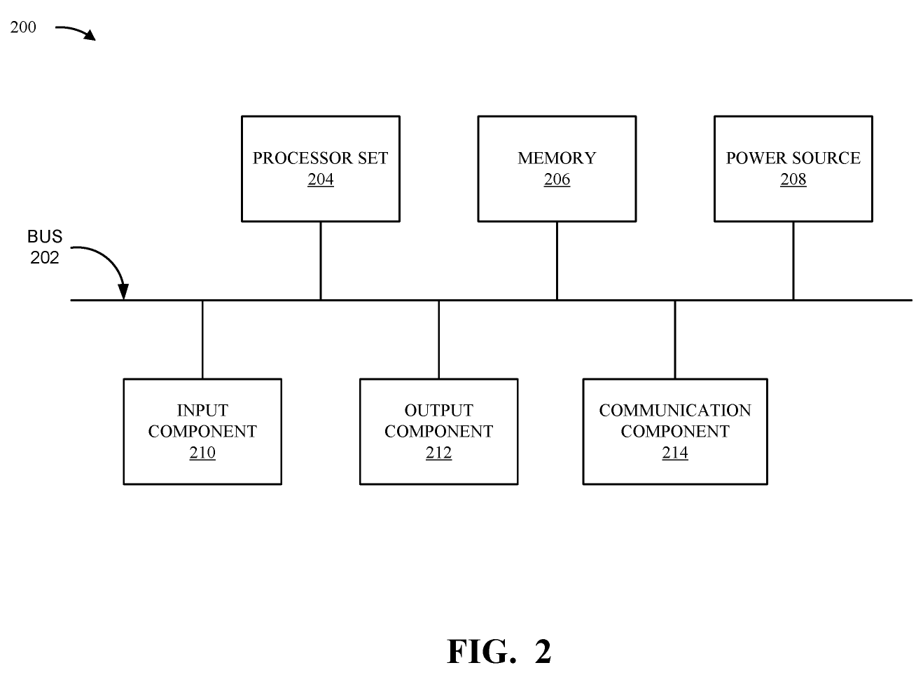

The control device 110 may be a component configured to manage the operations associated with the baby care device 102. The control device 110 may include one or more processors and memory, and may be, be similar to, include, or be included in the computing device 200 shown in FIG. 2. For example, the control device 110 may execute software and firmware to perform on-device machine-learning inference, process sensor data, and manage communication with the user 106 and the cloud system 104. In some implementations, the control device 110 is an embedded system, such as a microcontroller unit (MCU), optimized for low-power operation. In some implementations, the control device 110 is configured to receive user inputs via the display 112 and the microphone 114. The control device 110 may include an input component, such as a physical input button, and an output component, such as a display or a speaker. The control device 110 may be powered by the power source housed in the baby care device 102. In some implementations, the control device 110 is integrated with the baby care device 102.

The display 112 may be an output component configured to present visual information to the user 106. The display 112 may receive data from the control device 110 for presentation. For example, the display 112 may show an infant's vital signs, a patient risk score, a care recommendation, or the status of the baby care device 102. In some implementations, the display 112 is a liquid crystal display (LCD), a light-emitting diode (LED) display, or a touchscreen interface that functions as an input component.

The microphone 114 may be a sensor configured to capture audio from the environment 100. The microphone 114 may convert sound waves into an electrical signal that is then digitized to create an audio stream for processing by the control device 110. For example, the microphone 114 may be configured to capture an audio stream including the voice 116 of the user 106, infant vocalizations, or ambient background noise. In some implementations, the baby care device 102 may include an array of multiple microphones to facilitate functionalities such as noise cancellation or source localization.

The voice 116 represents an audible utterance from the user 106. The voice 116 may be captured by the microphone 114 of the baby care device 102. For example, the voice 116 may contain a wakeword to activate the baby care device 102, followed by a user command. The baby care device 102 may be configured to process the captured audio of the voice 116 on-device to identify the user command and generate an appropriate response. In some cases, the processing may rely on a machine-learning model stored in the baby care device 102. Although not shown in FIG. 1, the voice 116 may interact with the user 106 via other components of the baby care device 102, such as a speaker or a display.

FIG. 2 is a block diagram of an example internal configuration of a computing device 200 configured to perform functions described herein. The computing device 200 may be, be similar to, include, or be included in an apparatus for performing one or more methods, processes, algorithms, operations, tasks, and/or techniques, as described herein. The computing device 200 may be, be similar to, include, or be included in, the baby care device 102, the cloud system 104, and/or the control device 110, as shown in FIG. 1, among other examples. The computing device 200 includes a bus 202 that interconnects various components or units, such as a processor set 204, a memory 206, a power source 208, an input component 210, an output component 212, and a communication component 214, among other examples. One or more of the memory 206, the power source 208, the input component 210, the output component 212, or the communication component 214 can communicate with the processor set 204 via the bus 202.

The processor set 204 includes one or more processors. For example, the processor set 204 may be a central processing unit, such as a microprocessor, and may include single or multiple processors having single or multiple processing cores. The processor set 204 may include another type of device, or multiple devices, configured for manipulating or processing information. For example, the processor set 204 may include multiple processors interconnected in one or more manners, including hardwired or networked. The operations of the processor set 204 may be distributed across multiple devices or units that can be coupled directly or across a local area or other suitable type of network. The processor set 204 may include a cache, or cache memory, for local storage of operating data or instructions. The processor set 204 is implemented in hardware, firmware, or a combination of hardware and software. In some implementations, the processor set 204 includes one or more processors capable of being programmed to perform a function.

The processor set 204 may include one or more chiplets, chips, system-on-chips (SoCs), network-on-chips (NoCs), chipsets, packages, or devices that individually or collectively constitute or include the processor set. The processor set may include a processor (or “processing”) circuitry in the form of one or multiple processors, microprocessors, processing units (such as CPUs), GPUs, neural processing units (NPUs) and/or digital signal processors (DSPs)), processing blocks, application-specific integrated circuits (ASIC), programmable logic devices (PLDs) (such as field programmable gate arrays (FPGAs)), or other discrete gate or transistor logic or circuitry (all of which may be generally referred to herein individually as “one or more processors” or collectively as “the processor” or “the processor set”).

One or more of the processors of the processor set 204 may be individually or collectively configurable or configured to perform various operations described herein. In some implementations, a single processor may perform all of the operations described as being performed by the one or more processors. In some implementations, a group of processors collectively configurable or configured to perform a set of operations may include a first set of (one or more) processors configurable or configured to perform a first operation of the set and a second processor configurable or configured to perform a second operation of the set, or may include the group of processors all being configured or configurable to perform the set of operations. The first set of processors and the second set of processors may be the same set of processors or may be different sets of processors.

The memory 206 includes one or more memory components, which may each be volatile memory or non-volatile memory, that individually or collectively constitute a memory system. The memory system may include memory circuitry in the form of one or more memory devices, memory blocks, memory elements or other discrete gate or transistor logic or circuitry, each of which may include tangible storage media such as random-access memory (RAM) or read-only memory (ROM), or combinations thereof (all of which may be generally referred to herein individually as “memories” or collectively as “the memory,” “the memory system,” or “the memory circuitry”). The memory 206 may include non-transitory memory, transitory memory, or a combination thereof. Volatile memory may include RAM (e.g., a dynamic RAM (DRAM) module, such as a double data rate (DDR) synchronous DRAM (SDRAM)). Non-volatile memory may include a disk drive, a solid state drive, flash memory, or phase-change memory. In some implementations, the memory 206 may be distributed across multiple devices. For example, the memory 206 may include network-based memory or memory in multiple clients or servers performing the operations of those multiple devices. The memory 206 may be referred to as one or more computer-readable media. A computer-readable medium may include any storage unit (or multiple storage units) that store data or instructions that are readable by a processing system. A computer-readable medium may include, for example, at least one of a data repository, a data storage unit, a computer memory, a hard drive, a disk, or a random access memory.

One or more of the memories may be coupled (for example, operatively coupled, communicatively coupled, electronically coupled, or electrically coupled) with one or more of the processors of the processor set 204 and may individually or collectively store processor-executable instructions (e.g., code such as software) that, when executed by one or more of the processors, may configure or otherwise cause one or more of the processors to perform various functions or operations described herein. “Software” shall be construed broadly to mean instructions, instruction sets, code, code segments, program code, programs, subprograms, software modules, applications, software applications, software packages, routines, subroutines, objects, executables, threads of execution, procedures, and/or functions, among other examples, whether referred to as software, firmware, middleware, microcode, hardware description language, or otherwise.

In some implementations, the executable instructions may include application data or an operating system, among other examples. The executable instructions may include one or more application programs, which may be loaded or copied, in whole or in part, from non-volatile memory to volatile memory to be executed by the processor set 204. For example, the executable instructions may include instructions for performing techniques described in this disclosure. In some implementations, the application data may include functional programs, such as computational programs, analytical programs, or database programs, among other examples. The operating system may be, for example, Microsoft Windows®, Mac OS X®, or Linux®; an operating system for a mobile device, such as a smartphone or tablet device; or an operating system for a non-mobile device, such as a mainframe computer.

Reference to “one or more memories” should be understood to refer to any one or more memories of a corresponding device, such as the memory described in connection with FIG. 2. For example, operation described as being performed by, or data described as being stored on, one or more memories can be performed by, or stored on, respectively, the same subset of the one or more memories or different subsets of the one or more memories. Additionally or alternatively, in some examples, one or more of the processors may be preconfigured to perform various functions or operations described herein without requiring configuration by software. For example, the memory 206 may include data or instructions that are hard-wired into the processing system.

In the description herein, language describing a system, an apparatus, or a device as taking an action (such as performing, determining, initiating, receiving, calculating, deciding, computing, processing, etc.) is to be understood as describing that some appropriate component of the system, apparatus, or device is taking the action. As used herein, the term “component” is intended to be broadly construed as hardware and/or a combination of hardware and software.

An “engine” refers to a component constructed, programmed, configured, or otherwise adapted to perform a specific function or set of functions. The term engine as used herein means a tangible device, component, or arrangement of components implemented using hardware, such as by an ASIC or field-programmable gate array (FPGA), for example, or as a combination of hardware and software, such as by a processor-based computing platform and a set of program instructions that transform the computing platform into a special-purpose device to implement the particular functionality. An engine may also be implemented as a combination of the two, with certain functions facilitated by hardware alone, and other functions facilitated by a combination of hardware and software.

In an example, the software may reside in executable or non-executable form on a tangible machine-readable storage medium. Software residing in non-executable form may be compiled, interpreted, translated, or otherwise converted to an executable form prior to, or during, runtime. In an example, the software, when executed by the underlying hardware of the engine, causes the hardware to perform the specified operations. Accordingly, an engine is physically constructed, or specifically configured (e.g., hardwired), or temporarily configured (e.g., programmed) to operate in a specified manner or to perform part or all of any operations described herein in connection with that engine.

Considering examples in which engines are temporarily configured, each of the engines may be instantiated at different moments in time. For example, where the engines include a general-purpose hardware processor core configured using software, the general-purpose hardware processor core may be configured as respective different engines at different times. Software may accordingly configure a hardware processor core, for example, to constitute a particular engine at one instance of time and to constitute a different engine at a different instance of time.

In certain implementations, at least a portion, and in some cases, all, of an engine may be executed on the processor(s) of one or more computers that execute an operating system, system programs, and application programs, while also implementing the engine using multitasking, multithreading, distributed (e.g., cluster, peer-peer, cloud, etc.) processing where appropriate, or other such techniques. Accordingly, each engine may be realized in a variety of suitable configurations, and should generally not be limited to any particular implementation exemplified herein, unless such limitations are expressly called out. As used herein, the term “model” encompasses its plain and ordinary meaning. A model may include, among other things, one or more engines which receive an input and compute an output based on the input.

The power source 208 provides power to the computing device 200. For example, the power source 208 may be an interface to an external power distribution system. In an example, the power source 208 may be a battery, such as where the computing device 200 is a mobile device or is otherwise configured to operate independently of an external power distribution system. In some implementations, the computing device 200 may include or otherwise use multiple power sources. In some such implementations, the power source 208 can be a backup battery.

The input component 210 and/or the output component 212 may include one or more input interfaces and/or output interfaces configured for facilitating communication between the computing device 200 and one or more peripheral devices such as, for example, one or more sensors, detectors, displays, input devices, or other devices configured for facilitating interaction with the computing device 200 or the environment around the computing device 200. An input device may, for example, include a positional input device, such as a mouse, touchpad, touchscreen, or the like; a keyboard; or another suitable human or machine interface device. An output device may, for example, include a display, such as a liquid crystal display, a cathode-ray tube, a light emitting diode display, or other suitable display. In some implementations, the peripherals devices may include a geolocation component, such as a GPS location unit. In some examples, the peripheral devices may include a temperature sensor for measuring temperatures of components of the computing device 200, such as the processor set 204.

The communication component 214 may include an interface for facilitating a connection or link to a network. The communication component 214 may include a wired network interface or a wireless network interface. The computing device 200 may communicate with other devices via the communication component 214 using one or more network protocols, such as using Ethernet, TCP, IP, power line communication, an IEEE 802.X protocol (e.g., Wi-Fi, Bluetooth, or ZigBee), infrared, visible light, general packet radio service (GPRS), global system for mobile communications (GSM), code-division multiple access (CDMA), Z-Wave, a cellular communication protocol, another protocol, or a combination thereof. For example, the computing device 200 can communicate with a database server.

The communication component 214 may include a transceiver, which may include a transmitter or a receiver. In some configurations, one or a combination of antenna(s), modem(s), multiple input multiple output (MIMO) detectors, receive processors, transmit processors, and/or the transmit MIMO processors may be included in the transceiver. The transceiver may be under control of or used by one or more processors, and in some aspects in conjunction with processor-readable code stored in the memory, to perform aspects of the methods, processes, techniques, and/or operations described herein.

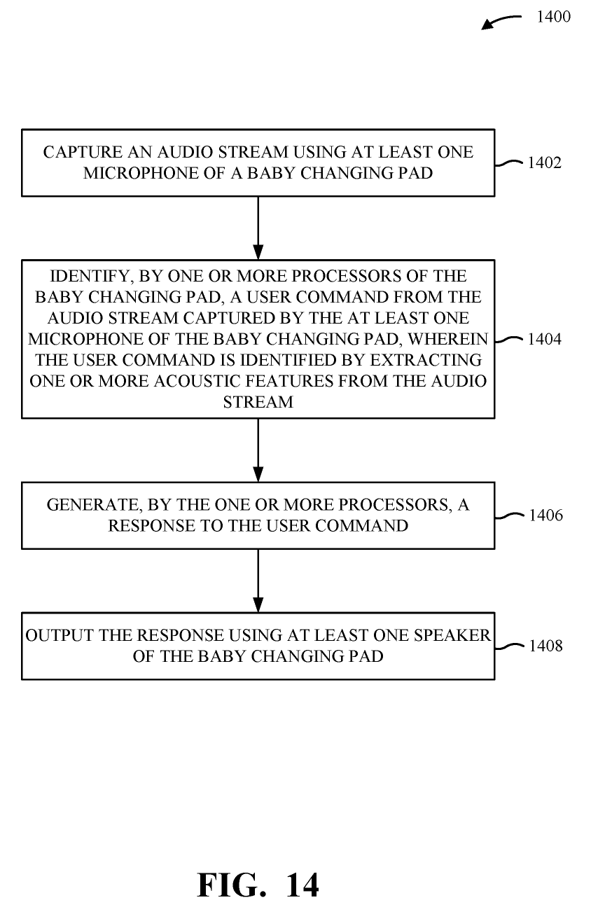

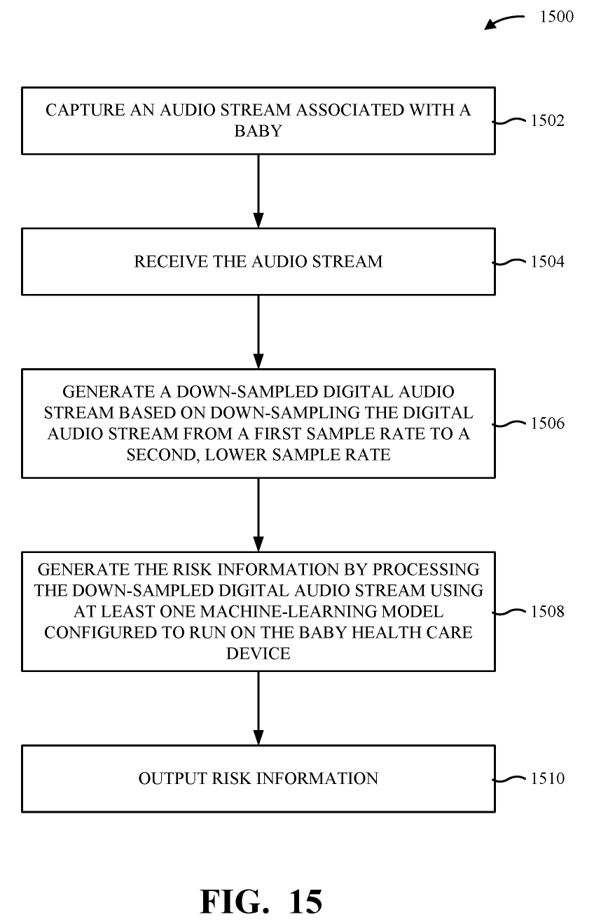

The processor set 204 may implement one or more techniques or perform one or more operations associated with on-device machine-learning processing, as described in more detail elsewhere herein. For example, the processor set 204 may perform or direct operations of, for example, technique 1400 of FIG. 14, technique 1500 of FIG. 15, or other techniques as described herein (alone or in conjunction with one or more other processors). The memory 206 may store data and program codes for the computing device 200. In some examples, the memory 206 may include a non-transitory computer-readable medium storing a set of instructions (for example, code or program code). The memory 206 may include one or more memories, such as a single memory or multiple different memories (of the same type or of different types). For example, the set of instructions, when executed (for example, directly, or after compiling, converting, or interpreting) by the processor set 204, may cause the processor to cause the computing device 200 to perform technique 1400 of FIG. 14, technique 1500 of FIG. 15 or other techniques as described herein. In some examples, executing instructions may include running the instructions, converting the instructions, compiling the instructions, and/or interpreting the instructions, among other examples.

The number and arrangement of components shown in FIG. 2 are provided as an example. The computing device 200 may include additional components, fewer components, different components, or differently arranged components than those shown in FIG. 2. Additionally, or alternatively, a set of components (e.g., one or more components) of the computing device 200 may perform one or more functions described as being performed by another set of components of the computing device 200.

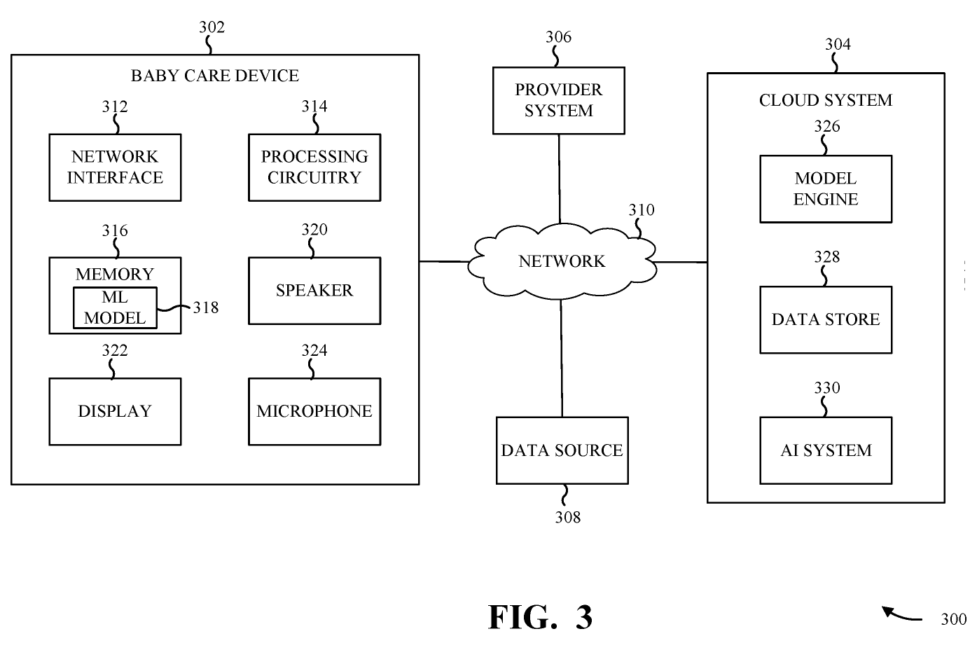

FIG. 3 is a block diagram of an example operating environment 300 associated with a baby care device 302. The operating environment 300 depicts the baby care device 302 in communication with a cloud system 304, a provider system 306, and a data source 308, via a network 310. In some implementations, one or more of the components of the operating environment 300 may be implemented using a computing device, such as the computing device 200 shown in FIG. 2.

The baby care device 302 may be an electronic apparatus designed to assist in monitoring, caring for, or analyzing an infant's well-being. The baby care device 302 may be, be similar to, include, or be included in the baby care device 102 shown in FIG. 1. For example, the baby care device 302 may be configured to perform on-device machine-learning processing using one or more embedded models, capture sensor data, and interact with external systems such as the cloud system 304.

In some implementations, the baby care device 302 is a resource-constrained device, such as a smart changing pad, equipped with a low-power microcontroller unit. The baby care device 302 may be configured to execute a specialized, lightweight neural network model to perform inference operations locally, thereby reducing latency and enhancing data privacy. For example, the baby care device 302 may process an audio stream to identify a wakeword or user command without transmitting audio data to an external server.

The cloud system 304 may be a remote computing environment providing services and resources over the network 310. The cloud system 304 may be, be similar to, include, or be included in the cloud system 104 shown in FIG. 1. For example, the cloud system 304 may be configured to receive data from the baby care device 302, train or refine machine-learning models, and provide responses or updated models back to the baby care device 302. In some implementations, the cloud system 304 hosts one or more trained machine-learning models that perform one or more operations described herein. The cloud system 304 may be implemented using one or more computing devices, such as the computing device 200 shown in FIG. 2. In some examples, the components of the cloud system 304 may be implemented in the cloud system 304 as services or microservices.

In some implementations, the cloud system 304 facilitates a hybrid processing architecture. For instance, if the baby care device 302 identifies a user command that cannot be fulfilled using on-device resources, the baby care device 302 may transmit data to the cloud system 304 for processing by other computational resources, such as a large language model. The cloud system 304 may then generate a response and transmit it back to the baby care device 302.

The provider system 306 may be a computing system associated with a healthcare provider, a hospital, or a clinical research organization. For example, the provider system 306 may be configured to receive physiological data or health risk assessments generated by the baby care device 302 or the cloud system 304. This may facilitate in-home monitoring of infants. In some implementations, the provider system 306 may be or include an electronic medical record (EMR) system or an electronic health record (EHR) system. For example, the provider system 306 may be configured to receive data recorded or generated by the baby care device 302 and store the data within an electronic health record. The provider system 306 may be implemented using one or more computing devices, such as the computing device 200 shown in FIG. 2. In some implementations, the provider system 306 may be operated by medical professionals who use the data to monitor patient progress, detect potential health issues, or conduct clinical studies. The provider system 306 may communicate with the baby care device 302 and the cloud system 304 via the network 310 to access and analyze infant health data.

The data source 308 may be a repository of data that may be used to train, augment, or validate machine-learning models. For example, the data source 308 may include datasets of infant vocalizations, background noise samples from nursery environments, physiological measurements, or clinical data. The cloud system 304 may access the data source 308 to augment training datasets for creating specialized machine-learning models. In some implementations, the data source 308 may be a publicly-accessible database. In some implementations, the data source 308 may be a repository associated with a clinical research organization that collects health data from clinical trials. In some implementations, the data source 308 may include another baby care device.

In some implementations, the data source 308 may be a third-party database, an internal data lake, or a collection of publicly available datasets. For example, to train a robust wakeword detection model, the cloud system 304 may combine synthetic speech samples with noise samples, such as baby cry audio or respiratory noise audio, obtained from the data source 308 to create an augmented training dataset.

The network 310 may be a communication network that facilitates data exchange between the baby care device 302, the cloud system 304, the provider system 306, and the data source 308. For example, the network 310 may be the Internet, a local area network (LAN), a wide area network (WAN), a cellular network, or a combination thereof. The network 310 may support various communication protocols, such as transport control protocol/Internet protocol (TCP/IP), hypertext transfer protocol (HTTP), real-time transport protocol (RTP), real-time transport protocol control protocol (RTCP), real-time transport protocol packet mode (RTP/RTCP), secure hypertext transport protocol (HTTPS), user datagram protocol (UDP), session initiation protocol (SIP), or any other suitable communication protocol.

In some implementations, the baby care device 302 may connect to the network 310 using a wireless communication component, such as a Wi-Fi or Bluetooth module. This connection may be used to transmit data to the cloud system 304 for processing or to receive software updates and new machine-learning models.

As shown in FIG. 3, the baby care device 302 includes a network interface 312, processing circuitry 314, a memory 316, a speaker 320, a display 322, and a microphone 324. The memory 316 further includes an ML model 318. In some implementations, two or more of the network interface 312, the processing circuitry 314, the memory 316, the speaker 320, the display 322, and the microphone 324 may be integrated into a single component. For example, the processing circuitry 314 and the memory 316 may be part of a single system-on-chip (SoC). In some implementations, one or more of the network interface 312, the processing circuitry 314, the memory 316, the speaker 320, the display 322, and the microphone 324 may be implemented using more than one computing device. For example, the processing circuitry 314 may include a host processor and a sound processor, either or both of which may be implemented by a computing device separate from the memory 316.

The network interface 312 may be a component configured to facilitate communication over the network 310. The network interface 312 may be, be similar to, include, or be included in the communication component 214 shown in FIG. 2. For example, the network interface 312 may be a wireless transceiver that supports protocols such as Wi-Fi or Bluetooth.

The processing circuitry 314 may be configured to execute instructions and perform computations for the baby care device 302. The processing circuitry 314 may be, be similar to, include, or be included in the processor set 204 shown in FIG. 2. For example, the processing circuitry 314 may be a low-power MCU configured to perform on-device inference using the ML model 318. In some implementations, the processing circuitry 314 may include a central processing unit (CPU), a graphic processing unit (GPU), a neural processing unit (NPU), a tensor processing unit (TPU), or an FPGA. The memory 316 may be a component configured to store data and instructions for the baby care device 302. The memory 316 may be, be similar to, include, or be included in the memory 206 shown in FIG. 2. For example, the memory 316 may be a combination of volatile memory, such as RAM, and non-volatile memory, such as flash memory, which stores the ML model 318 and firmware for the device.

The ML model 318 may be a machine-learning model stored in the memory 316 and configured to run on the processing circuitry 314. For example, the ML model 318 may be a neural network model, such as an LSTM model or a DNN model, that has been optimized for execution on resource-constrained hardware. The ML model 318 may be trained to identify user commands, wakewords, or infant vocalizations from an audio stream captured by the microphone 324.

The speaker 320 may be an output component configured to produce sound. The speaker 320 may be, be similar to, include, or be included in the output component 212 shown in FIG. 2. For example, the speaker 320 may be used to provide an audible response to a user command, play sounds for an infant, or generate alerts. The display 322 may be an output component configured to present visual information. The display 322 may be, be similar to, include, or be included in the display 112 shown in FIG. 1. For example, the display 322 may show an infant's physiological data, a device status, or a care recommendation.

The microphone 324 may be an input component configured to capture audio. The microphone 324 may be, be similar to, include, or be included in the microphone 114 shown in FIG. 1. For example, the microphone 324 may capture an audio stream that is processed on-device by the processing circuitry 314 using the ML model 318 to identify user commands. Although not shown in FIG. 3, the baby care device 302 may include one or more sensors, such as a temperature sensor or a heart rate monitor, to obtain physiological measurements of an infant. In some implementations, one or more of the speaker 320, the display 322, and the microphone 324 may be integrated into a single component.

As shown in FIG. 3, the cloud system 304 includes a model engine 326, a data store 328, and an AI system 330. In some implementations, one or more of the model engine 326, the data store 328, and the AI system 330 may be implemented as distributed services running on one or more servers. For example, the components may be deployed as microservices in a cloud computing environment.

The model engine 326 may be a component configured to create, train, or optimize machine-learning models. For example, the model engine 326 may implement a process that generates synthetic training data, augments the data with noise samples from the data store 328, and trains a neural network model. The model engine 326 may then provide a trained model for deployment on the baby care device 302.

The data store 328 may be a component configured to store data used by the cloud system 304. For example, the data store 328 may store physiological data received from the baby care device 302, training datasets for machine-learning models, or user account information. The model engine 326 may access the data store 328 to retrieve data for model training. The data store 328 may be, be similar to, include, or be included in the memory 206 shown in FIG. 2. For example, the data store 328 may be a non-transitory, computer-readable medium. The data store 328 may include one or more data lakes, data warehouses, or relational database management system (RDBMS). The cloud system 304 may access the data store 328 to retrieve information for model training or data augmentation.

The AI system 330 may be a component configured to perform artificial intelligence processing. For example, the AI system 330 may include one or more large language models (LLMs), risk assessment models, or other AI tools that use more computational resources than are available on the baby care device 302. In a hybrid architecture, the baby care device 302 may transmit data to the AI system 330 to handle complex queries, and the AI system 330 may generate a response to be sent back to the baby care device 302.

In some implementations, the operating environment 300 may facilitate a comprehensive baby care ecosystem. The integration of the on-device capabilities of the baby care device 302 with the computational resources of the cloud system 304 may facilitate a responsive and private monitoring solution. For example, user commands or initial data processing may be handled on the baby care device 302 using the embedded ML model 318, which may facilitate low-latency responses and maintain data, such as audio captured by the microphone 324, on the device. This on-device processing may maintain functionality even with intermittent network connectivity.

In some implementations, the hybrid nature of the operating environment 300 may facilitate sophisticated analysis and personalization. For instance, physiological data gathered by the baby care device 302 may be transmitted to the cloud system 304, where the model engine 326 may leverage large datasets from the data store 328 and the data source 308 to train and refine risk-assessment models. These models, tailored to a specific infant's health profile, may then be deployed back to the baby care device 302 as an updated ML model 318. This may create a continuous learning loop where the system becomes progressively more attuned to the individual needs of the infant.

The inclusion of the provider system 306 in the operating environment 300 extends the utility of the system into clinical settings. For example, if the on-device or cloud-based analysis identifies a potential health risk, such as a pattern indicative of respiratory distress or failure to thrive, the system may be configured to securely transmit relevant data and alerts to the provider system 306. This may facilitate remote monitoring of infants by healthcare professionals, review of objective data, and proactive intervention.

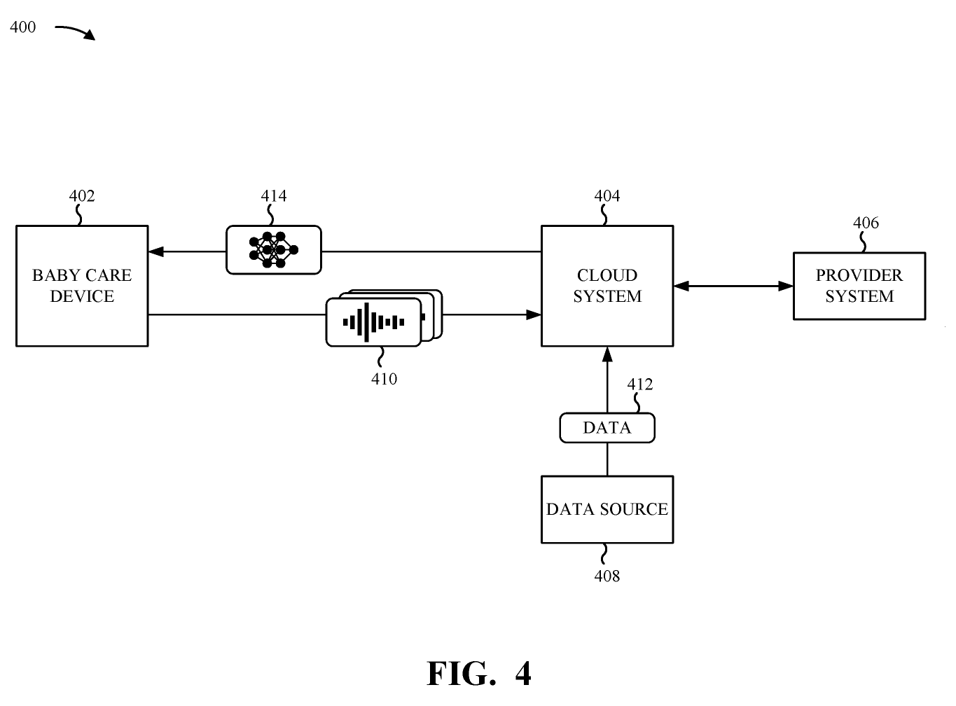

FIG. 4 is a data flow diagram 400 of an example associated with on-device machine-learning processing for baby care devices. The data flow diagram 400 illustrates the flow of data and models within a system connecting a baby care device 402, a cloud system 404, a provider system 406, and a data source 408. The baby care device 402 may initiate a data flow by transmitting an ML model 414 to the cloud system 404, and the cloud system 404 may interact with an audio stream 410, data 412, the data source 408, and the provider system 406.

The baby care device 402 may be an electronic apparatus designed to assist in monitoring or caring for an infant. The baby care device 402 may be, be similar to, include, or be included in the baby care device 102 shown in FIG. 1 or the baby care device 302 shown in FIG. 3. For example, the baby care device 402 may be configured to perform on-device processing of audio commands and physiological data using one or more machine-learning models. In some implementations, the baby care device 402 is a resource-constrained device, such as a smart changing pad, that executes a lightweight neural network model to provide low-latency, private, and reliable operation. The baby care device 402 may transmit data, such as physiological measurements or an existing ML model 414, to the cloud system 404 for processing, analysis, or model refinement.

In some implementations, the baby care device 402 is configured to operate as part of a hybrid processing architecture. For example, while certain operations such as wakeword detection may be performed locally, other tasks may be offloaded to the cloud system 404. The baby care device 402 may determine that a user command cannot be fulfilled with on-device resources and, in response, transmit data to the cloud system 404 to leverage other computational resources, such as a large language model. In some implementations, the baby care device 402 may transmit raw audio data, extracted acoustic features, or text generated by an on-device speech-to-text engine.

The cloud system 404 may be a remote computing environment that provides computational resources, data storage, and services accessible over a network. The cloud system 404 may be, be similar to, include, or be included in the cloud system 104 shown in FIG. 1 or the cloud system 304 shown in FIG. 3. For example, the cloud system 404 may be configured to receive data from the baby care device 402, train or refine machine-learning models, and provide responses or updated models back to the baby care device 402. In some implementations, the cloud system 404 is configured to receive an existing ML model 414 from the baby care device 402 and retrain or update the ML model 414 using additional data, such as an audio stream 410 or data 412 from the data source 408. The cloud system 404 may be configured to communicate with a provider system 406 to share health-related data or risk assessments.

In some implementations, the cloud system 404 may host a model engine configured to create specialized neural network models for deployment on the baby care device 402. This process may include generating synthetic audio samples, augmenting them with noise samples specific to an infant care environment (e.g., baby cry audio, respiratory noise), and training a lightweight model optimized for resource-constrained hardware. For example, the cloud system 404 may generate a personalized model for a specific infant by training the personalized model on that infant's physiological data, and then deploy the trained model to the baby care device 402.

The provider system 406 may be a computing system associated with a healthcare provider, a hospital, or a clinical research organization. The provider system 406 may be, be similar to, include, or be included in the provider system 306 shown in FIG. 3. For example, the provider system 406 may be configured to receive physiological data, health risk assessments, or alerts generated by the cloud system 404 based on data from the baby care device 402. In some implementations, the provider system 406 may include an EHR system that stores and manages infant health data, which may facilitate remote monitoring by healthcare professionals. The bidirectional communication between the cloud system 404 and the provider system 406 may facilitate the exchange of clinical data, care recommendations, and patient updates.

The data source 408 may be a repository of data that may be used to train, augment, or validate machine-learning models. The data source 408 may be, be similar to, include, or be included in the data source 308 shown in FIG. 3. For example, the data source 408 may include datasets of infant vocalizations, background noise samples from nursery environments, physiological measurements, or clinical data from third-party sources. In some implementations, the cloud system 404 may access the data source 408 to retrieve data for augmenting training datasets, which may enhance the robustness and accuracy of the machine-learning models deployed on the baby care device 402. For example, to train a wakeword detection model, the cloud system 404 may combine synthetic speech with baby cry audio or respiratory noise audio from the data source 408.

The audio stream 410 may represent digital audio data that is processed by the cloud system 404. The audio stream 410 may originate from the baby care device 402 or another source. For example, the audio stream 410 may include user voice commands, infant vocalizations, or ambient sounds captured in a nursery. In some implementations, the cloud system 404 may use the audio stream 410 as part of a training dataset to create or refine a machine-learning model. For instance, the audio stream 410 may be used as a source of noise samples for data augmentation, which may improve the model's performance in real-world environments.

The data 412 may represent various forms of information used by the cloud system 404. For example, the data 412 may include physiological measurements collected by the baby care device 402, such as weight, temperature, or heart rate. In some implementations, the data 412 may include training data, model parameters, or user-specific information stored within the cloud system 404. The cloud system 404 may use the data 412 in conjunction with data from the data source 408 and the audio stream 410 to perform model training, risk assessment, or other analytical tasks. For example, the data 412 and audio stream 410 may be used to train a machine-learning model 414 that may be deployed on the baby care device 402 to automate infant care. As shown, the cloud system 404 may transmit the ML model 414 back to the baby care device 402. In some implementations, the cloud system 404 may update an existing ML model 414 on the baby care device 402.

The ML model 414 may be a machine-learning model, such as a neural network model, that is transmitted from the baby care device 402 to the cloud system 404. The ML model 414 may be, be similar to, include, or be included in the ML model 318 shown in FIG. 3. For example, the ML model 414 may be a personalized model that has been running on the baby care device 402. In some implementations, the ML model 414 may be transmitted to the cloud system 404 for retraining or updating based on new data. This may facilitate a continuous learning loop where the model is periodically refined to improve its performance or adapt to changes in an infant's health profile. After refinement, an updated version of the ML model 414 may be deployed back to the baby care device 402.

In some implementations, the data flow diagram 400 may illustrate a continuous learning and personalization loop for an infant care system. For example, the baby care device 402 may collect physiological data 412 over time. This data 412, along with the current version of the ML model 414 running on the device, may be transmitted to the cloud system 404. The cloud system 404 may use this new data 412, potentially augmented with audio streams 410 and additional information from the data source 408, to retrain and refine the ML model 414, creating a version that is more personalized to the specific infant. The updated ML model 414 is then transmitted back to the baby care device 402, enhancing its on-device analytical capabilities. This process may facilitate a system that adapts to an infant's individual growth and health patterns. The cloud system 404 may also share derived insights or alerts with the provider system 406 to facilitate proactive medical care.

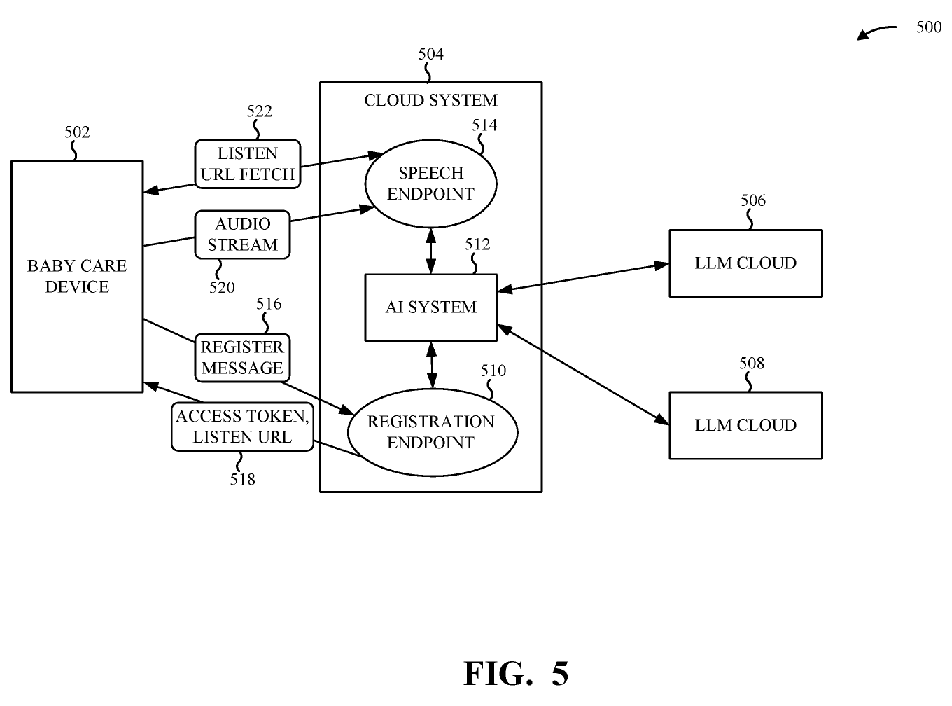

FIG. 5 is a data flow diagram of another example associated with on-device machine-learning processing for baby care devices. The data flow diagram 500 illustrates a hybrid processing architecture wherein a baby care device 502 may interact with a cloud system 504 to handle complex queries that extend beyond the capabilities of its on-device machine-learning models. This interaction may include a registration process to establish a secure session and a subsequent data exchange to process an audio stream 520 and retrieve a generated response. The cloud system 504 may, in some implementations, leverage the computational resources of one or more external large language model (LLM) clouds, such as an LLM cloud 506 and an LLM cloud 508.

The baby care device 502 may be an electronic apparatus designed to assist in monitoring or caring for an infant. The baby care device 502 may be, be similar to, include, or be included in the baby care device 102 shown in FIG. 1, the baby care device 302 shown in FIG. 3, or the baby care device 402 shown in FIG. 4. For example, the baby care device 502 may be configured to perform on-device processing of audio commands using one or more embedded machine-learning models. In some implementations, the baby care device 502 is a resource-constrained device, such as a smart changing pad, configured to execute a specialized, lightweight neural network model to provide low-latency and private operation.

In the context of the data flow diagram 500, the baby care device 502 may be configured to operate within a hybrid processing architecture. A processor set of the baby care device 502 may determine, based on an inference operation, that an identified user command cannot be fulfilled using on-device resources. For example, a user may ask a complex, open-ended question that requires the advanced natural language understanding capabilities of a large language model. In response to determining that the identified user command cannot be fulfilled using the on-device resources, the baby care device 502 may be configured to initiate a communication session with the cloud system 504 to offload the processing of the user command.

To facilitate this hybrid processing, the baby care device 502 may first transmit a register message 516 to the cloud system 504. The register message 516 may be a data structure transmitted from the baby care device 502 to the registration endpoint 510 of the cloud system 504. The register message 516 may be the initial communication sent by the baby care device 502 when it seeks to offload a query to the cloud. The purpose of the register message 516 may be to initiate a secure session and authenticate the device. The register message 516 may be formatted according to a predefined communication protocol. The register message 516 may include a unique device identifier, which may be used by the cloud system 504 to identify the specific baby care device 502 that is making the request. This may be used for logging, analytics, or personalization purposes. In some implementations, the register message 516 may include security credentials, such as a pre-shared key or a digital certificate, to prove the identity of the baby care device 502 and prevent unauthorized access to the cloud system 504. The register message 516 may contain metadata about the request, such as the type of query or the version of the software running on the baby care device 502.

After successfully registering, the baby care device 502 may receive an access token and listen URL 518. The access token and listen URL 518 may be a data structure transmitted from the registration endpoint 510 of the cloud system 504 to the baby care device 502 in response to a successful register message 516. This data structure may contain information for the baby care device 502 to proceed with the offloaded query processing. The access token portion of the access token and listen URL 518 may be a secure, often temporary, credential that the baby care device 502 may use to authenticate subsequent requests to the cloud system 504, such as the transmission of the audio stream 520. Using an access token may be more secure than repeatedly sending a static device identifier or password. The listen URL portion of the access token and listen URL 518 may be a unique and secure network address, such as a Uniform Resource Locator (URL), where the final response to the user's query will be made available. By providing a unique listen URL for each session, the cloud system 504 may maintain the privacy and integrity of the communication by having the baby care device 502 only retrieve the response intended for it.

The baby care device 502 may then transmit an audio stream 520, which may contain the user's complex query, to the cloud system 504 for analysis. The audio stream 520 may be a data structure representing the digital audio of a user's query that has been captured by the baby care device 502. The audio stream 520 may be transmitted from the baby care device 502 to the speech endpoint 514 of the cloud system 504 as part of the hybrid processing workflow. The audio stream 520 is transmitted after the baby care device 502 has determined that the query cannot be handled by its on-device resources.

The format of the audio stream 520 may vary depending on the specific implementation of the hybrid architecture. In some implementations, the audio stream 520 may be a raw, down-sampled digital audio stream, where the baby care device 502 performs minimal local processing before transmission. This approach may offload the maximum amount of processing to the cloud. In some implementations, to conserve network bandwidth, the audio stream 520 may not be raw audio but rather a more compact representation. For example, the baby care device 502 may first extract acoustic features, such as MFCCs, from the raw audio and the audio stream 520 may contain these extracted features instead of the full audio waveform.

After the cloud system 504 has processed the query and generated a response, the baby care device 502 may perform a listen URL fetch operation 522 to retrieve the response for output to a user, for instance, via at least one speaker. The listen URL fetch operation 522 may be an operation performed by the baby care device 502 to retrieve the final response to its query from the cloud system 504. This operation may be the final step in the hybrid processing data flow from the perspective of the baby care device 502. The listen URL fetch operation 522 may be initiated after the baby care device 502 has transmitted the audio stream 520 and after a period of time for the cloud system 504 to process the query and generate a response.

To perform the listen URL fetch operation 522, the baby care device 502 may make a network request, such as an HTTP GET request, to the unique listen URL that it received as part of the access token and listen URL 518 data structure. The request may include the access token for authentication. In response to a successful listen URL fetch operation 522, the cloud system 504 may transmit the final response back to the baby care device 502. The response may be in various formats, such as a text string or a synthesized audio file. The baby care device 502 may then process this response and provide it to the user, for example, by playing the audio file through its speaker.

The cloud system 504 may be a remote computing environment that provides computational resources, data storage, and services accessible over a network. The cloud system 504 may be, be similar to, include, or be included in the cloud system 104 shown in FIG. 1, the cloud system 304 shown in FIG. 3, or the cloud system 404 shown in FIG. 4. In the data flow diagram 500, the cloud system 504 is configured to act as an intermediary, receiving complex queries from the baby care device 502 and orchestrating their processing using other, external AI resources.

The cloud system 504 may include several functional components to manage the data flow. These components may include a registration endpoint 510, an AI system 512, and a speech endpoint 514. The registration endpoint 510 may be configured to handle initial communication and authentication from the baby care device 502. The speech endpoint 514 may be configured to receive the audio stream 520 containing the user query. The AI system 512 may be configured to coordinate the processing of the query, which may include interacting with one or more external large language models, such as the LLM cloud 506 and the LLM cloud 508. In some implementations, the AI system 512 may facilitate establishing a secure session with the baby care device 502 and one or more LLMs that are hosted specifically for that baby care device 502. For example, in some implementations, the cloud system 504 may establish unique endpoints associated with the LLM cloud 506 and the LLM cloud 508 that the baby care device 502 may access through the speech endpoint 514. In this way, the cloud system 504 may facilitate AI pipelines that are specific to baby care devices, users, or families.

After receiving a query from the baby care device 502, the cloud system 504 may process the request and generate a response. The AI system 512 may be configured to select an appropriate large language model from the LLM cloud 506 or the LLM cloud 508, transmit the processed query, and receive a generated response. The cloud system 504 may then make this response available at the specific network location identified by the listen URL that was provided to the baby care device 502 during the registration phase. In some implementations, the cloud system 504 may be configured to receive, from a cloud environment, a machine-learning model configured to run on the baby changing pad, wherein the machine-learning model is trained based on a set of data.

The LLM cloud 506 may be an external, remote computing environment that hosts a large language model. A large language model may be a complex neural network model trained on vast amounts of text and data, capable of understanding and generating human-like language. The computational and memory requirements for such models may necessitate their deployment in a cloud-based server environment rather than on a resource-constrained edge device. The LLM cloud 506 may be communicatively coupled to the AI system 512 within the cloud system 504.

The LLM cloud 506 may receive a processed query from the AI system 512 of the cloud system 504. This query may be in the form of a text string that was generated by a speech-to-text engine within the cloud system 504 after processing the audio stream 520. The large language model hosted by the LLM cloud 506 may then analyze the query, generate a relevant and contextually appropriate response, and transmit that response back to the AI system 512. The inclusion of the LLM cloud 506 as part of the overall architecture may facilitate a powerful and flexible user experience. While the baby care device 502 handles certain commands locally, the system may escalate other conversational queries to the LLM cloud 506 via the cloud system 504. This hybrid approach may combine the benefits of low-latency on-device processing with the advanced capabilities of large-scale AI models.

The LLM cloud 508 may be another external, remote computing environment that, similar to the LLM cloud 506, hosts a large language model. The presence of multiple, distinct LLM clouds, such as the LLM cloud 506 and the LLM cloud 508, may provide the system with redundancy, flexibility, or access to different specialized models. The AI system 512 of the cloud system 504 may be configured to select between the LLM cloud 506 and the LLM cloud 508 based on various criteria. For example, the AI system 512 may be configured to route queries to a specific LLM cloud based on the type of query, the current operational load of each LLM cloud, the cost associated with each service, or the geographic location of the user to reduce network latency. In some implementations, the LLM cloud 506 may host a general-purpose conversational model, while the LLM cloud 508 may host a model specialized in providing medical or child development information.

The AI system 512 may maintain a configuration that maps certain types of user commands or keywords to a preferred LLM cloud. For example, a query containing the word "sleep training" may be routed to the LLM cloud 508, while a query for a weather forecast may be routed to the LLM cloud 506. This intelligent routing may facilitate more accurate and relevant responses for the user. Similarly, the AI system 512 may be configured to determine a geographic location associated with the baby changing pad and select between the LLM cloud 506 and the LLM cloud 508 based on a location-based parameter. For example, the AI system 512 may route queries to the LLM cloud 508 when the geographic location of the device is within a threshold distance of a location of an adult user and route queries to the LLM cloud 506 when the geographic location of the device is within a threshold distance of a location of a child user.

The registration endpoint 510 may be a component within the cloud system 504 configured to manage the initiation of secure communication sessions with one or more baby care devices. The registration endpoint 510 may be implemented as a specific network interface, such as an application programming interface (API) endpoint, that listens for incoming connection requests. When the baby care device 502 determines that it needs to offload a query, it may first communicate with the registration endpoint 510.

The registration endpoint 510 may receive a register message 516 from the baby care device 502. This message may contain authentication credentials or a unique identifier for the baby care device 502. The registration endpoint 510 may then perform an authentication and authorization process to verify the identity of the baby care device 502. Upon successful authentication, the registration endpoint 510 may generate and transmit an access token and listen URL 518 back to the baby care device 502. The access token may be a secure, time-limited credential that the baby care device 502 may include in subsequent communications to prove its identity, while the listen URL may be a unique network address where the final response to the user's query will be made available. This process may establish a secure and stateful session for the hybrid processing operation.

The AI system 512 may be a component of the cloud system 504 configured to orchestrate the processing of offloaded user queries. The AI system 512 may be, be similar to, include, or be included in the AI system 330 shown in FIG. 3. The AI system 512 may be configured to receive data from other components within the cloud system 504, such as the registration endpoint 510 and the speech endpoint 514. For example, after the speech endpoint 514 processes the incoming audio stream 520, it may forward the resulting data (e.g., a text transcription) to the AI system 512. The AI system 512 may then perform additional processing, such as intent recognition or entity extraction, to format the query for a large language model.

The AI system 512 may be configured to manage communications with one or more external LLM clouds, such as the LLM cloud 506 and the LLM cloud 508. The AI system 512 may select an appropriate LLM, transmit the formatted query, receive the generated response, and then coordinate with other components to make that response available to the baby care device 502 at the designated listen URL. In some implementations, the AI system 512 may be configured to route queries to one or more LLMs based on various criteria. For example, the AI system 512 may route queries to a specific LLM based on the type of query or the complexity of the query. In some implementations, the AI system 512 may be configured to route queries to the LLM cloud 506 when the query cannot be processed locally and route queries to the LLM cloud 508 when the query can be processed locally. In some implementations, the AI system 512 may be configured to select between LLM clouds based on location, availability, or computational load. The AI system 512 may also be configured to route queries based on an associated geographic location of the user or a location of the baby changing pad. In some implementations, the AI system 512 may be configured to select between LLM clouds based on other factors such as, for example, model availability, computational resources, or system load.

The speech endpoint 514 may be a component within the cloud system 504 that is specifically configured to receive and process audio data. The speech endpoint 514 may be implemented as a network interface, such as an API endpoint, designed to handle streaming or file-based audio uploads. After the baby care device 502 has successfully registered with the registration endpoint 510, it may transmit the audio stream 520 to the speech endpoint 514. The speech endpoint 514 may be configured to perform initial audio processing tasks. In some implementations, the speech endpoint 514 may include a speech-to-text (STT) engine that converts the incoming audio stream 520 into a text string. This may be useful in scenarios where the baby care device 502 offloads the raw audio data, and the conversion to text is performed in the cloud.

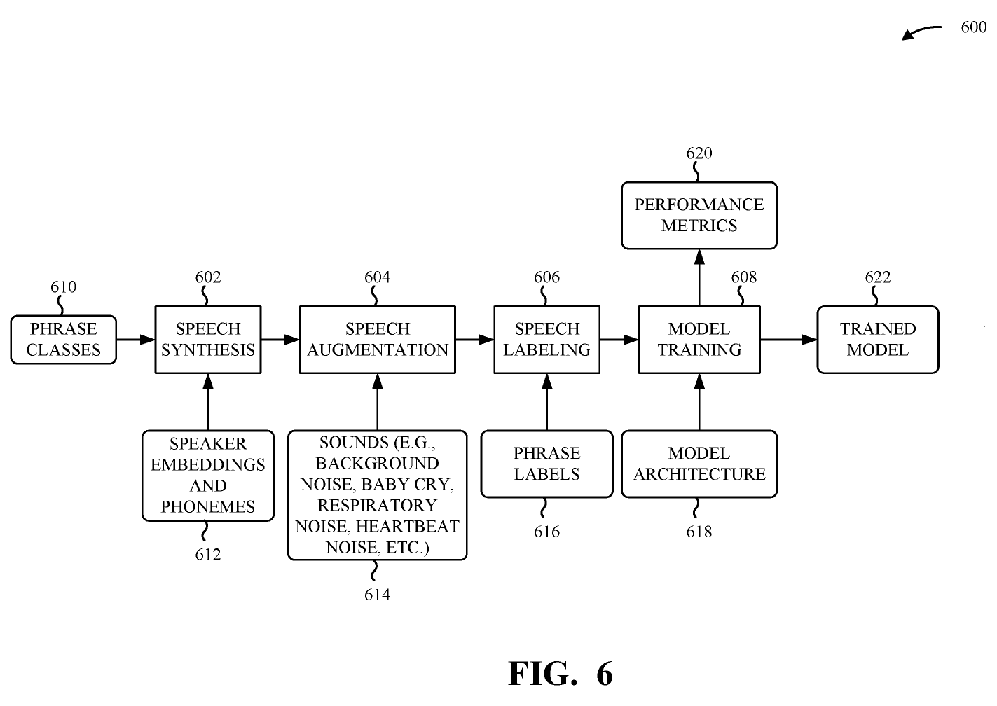

FIG. 6 is a flow diagram of an example process 600 associated with on-device machine-learning processing for baby care devices. The process 600 illustrates the creation of a specialized neural network model for a baby care device. The process 600 may begin with generating synthetic audio samples and augmenting them with environmental noise before training the model. The process 600 includes a speech synthesis 602 operation, a speech augmentation 604 operation, a speech labeling 606 operation, a model training 608 operation, phrase classes 610, speaker embeddings and phonemes 612, sounds 614, phrase labels 616, a model architecture 618, performance metrics 620, and a trained model 622. The process 600 may be implemented by a cloud system, such as the cloud system 304 shown in FIG. 3.

The process 600 may begin with the speech synthesis 602 operation. The speech synthesis 602 operation may include generating a first dataset of synthetic audio samples corresponding to one or more target phrases. For example, the speech synthesis 602 operation may be used to generate audio files of a wakeword, such as "Hey Woddle", spoken in various accents or tones. The speech synthesis 602 operation may include receiving phrase classes 610 and speaker embeddings and phonemes 612 as inputs. The output of the speech synthesis 602 operation may be a set of synthetic audio files that serve as the positive examples for training a machine-learning model.

The phrase classes 610 may be a data structure representing the text of the target phrases to be synthesized. For example, the phrase classes 610 may include a list of user commands (e.g., "turn on the light," "play music") or wakewords that the baby care device is intended to recognize. The speech synthesis 602 operation may involve using the phrase classes 610 as the textual basis for generating the corresponding audio. The speaker embeddings and phonemes 612 may be a data structure containing information used to control the characteristics of the synthesized speech. For example, speaker embeddings may represent the vocal characteristics of different speakers, which may be used to generate audio in various voices, while phonemes provide the phonetic breakdown of words, which may be used for pronunciation. The speech synthesis 602 operation may include using the speaker embeddings and phonemes 612 to create a diverse and realistic set of synthetic audio samples.

Following the speech synthesis 602 operation, the process 600 may proceed to the speech augmentation 604 operation. The speech augmentation 604 operation may include creating an augmented training dataset by combining the synthetic audio samples from the speech synthesis 602 operation with a second dataset of noise samples. This augmentation process is designed to make the resulting machine-learning model more robust in its target operational environment. For example, the speech augmentation 604 operation may involve mixing a synthesized wakeword with the sound of a baby crying to train the model to recognize the wakeword even in a noisy nursery. The speech augmentation 604 operation may include receiving the synthetic speech from the speech synthesis 602 operation and sounds 614 as inputs.

The sounds 614 may be a data structure representing a collection of audio samples used for data augmentation. The sounds 614 may be curated to include acoustic data relevant to an infant care environment. For example, the sounds 614 may include not only general background noise but also infant-related sounds such as baby cry audio, respiratory noise audio, or heart beating noise audio. By incorporating the sounds 614 into the training data, the speech augmentation 604 operation may be used to create a model that is less prone to false activations or missed detections in a real-world nursery setting. The output of the speech augmentation 604 operation is an augmented dataset of audio files ready for labeling.

The process 600 may then perform the speech labeling 606 operation. The speech labeling 606 operation may include associating each audio sample in the augmented dataset with a correct label or classification. For example, an audio file containing the synthesized wakeword mixed with background noise may be labeled as a positive example of the wakeword, while an audio file containing only background noise may be labeled as a negative example. The speech labeling 606 operation may include using phrase labels 616 to annotate the data. The phrase labels 616 may be a data structure that provides the ground-truth classifications for the training data. The phrase labels 616 may correspond to the phrase classes 610 and are used by the speech labeling 606 operation to assign the correct label to each audio sample. The output of the speech labeling 606 operation is a fully labeled, augmented training dataset.

The labeled dataset is then used in the model training 608 operation. The model training 608 operation may include training a neural network model using the augmented training dataset. The model training 608 operation may include using an iterative process where the model's parameters are adjusted to minimize the difference between its predictions and the ground-truth labels from the speech labeling 606 operation. The model architecture 618 provides the structural blueprint for the neural network being trained. The model architecture 618 may be a data structure that defines the type, number, and arrangement of layers in the neural network, such as LSTM layers, flatten layers, or sigmoid layers. The model architecture 618 may be designed to be lightweight and efficient for deployment on a resource-constrained device.

During the model training 608 operation, performance metrics 620 may be generated. The performance metrics 620 may be a data structure containing quantitative measurements of the model's performance, such as accuracy, precision, or recall. These metrics may be used to evaluate the training process and to determine if adjustments to the model architecture 618 or training parameters are needed. The final output of the model training 608 operation is a trained model 622. The trained model 622 is the optimized neural network model that has been trained on the environment-specific augmented data. The trained model 622 may be provided for deployment in an audio recognition application on a baby care device. In some implementations, the trained model 622 may be provided in a standard format like ONNX and subsequently compiled into embeddable C code for deployment on a microcontroller unit.

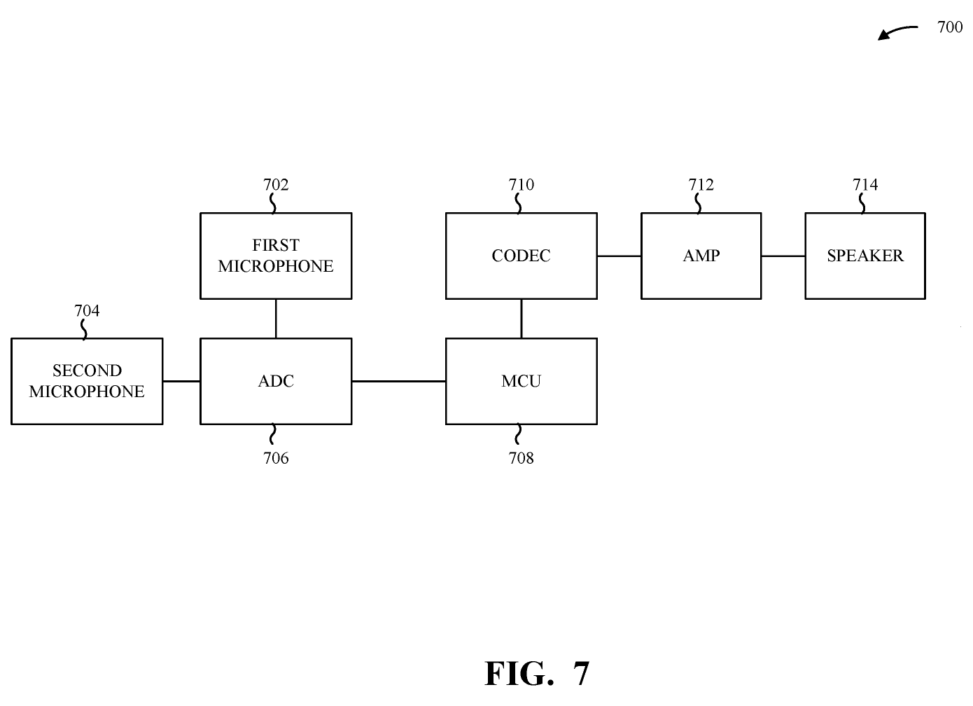

FIG. 7 is a block diagram of an example of an audio processing pipeline 700 of a baby care device. The audio processing pipeline 700 illustrates a hardware signal flow for audio input and output, including a first microphone 702, a second microphone 704, an ADC 706, a microcontroller unit 708, a codec 710, an amplifier 712, and a speaker 714. The audio processing pipeline 700 may be implemented by a baby care device, such as the baby care device 102 shown in FIG. 2.

The first microphone 702 may be a sensor configured to capture audio from the environment. The first microphone 702 may be, be similar to, include, or be included in the microphone 114 shown in FIG. 1 or the microphone 324 shown in FIG. 3. For example, the first microphone 702 may be configured to convert sound waves into an analog electrical signal. This signal may then be provided to the ADC 706 for digitization. In some implementations, the first microphone 702 is part of an array of microphones used to facilitate functionalities such as noise cancellation or sound source localization within a nursery environment. The frequency response of the first microphone 702 may be tailored to capture characteristics of both an adult's speech and an infant's vocalizations. In some implementations, the first microphone 702 may be one of multiple microphones placed on a baby care device to create a stereo or multi-channel audio input.

This arrangement, including the first microphone 702 and the second microphone 704, may be used to enhance the performance of on-device audio processing algorithms. For example, by comparing the signals from the first microphone 702 and the second microphone 704, the microcontroller unit 708 may be configured to suppress background noise and identify a user's voice command. The second microphone 704 may be another sensor configured to capture audio from the environment, operating in conjunction with the first microphone 702. The second microphone 704 may be, be similar to, include, or be included in the microphone 114 shown in FIG. 1. For example, the second microphone 704 may capture a second channel of audio to create a stereo input, which is then provided to the ADC 706. In some implementations, the second microphone 704 is identical in specification to the first microphone 702 to provide for balanced audio capture.

The second microphone 704 may operate with the first microphone 702 to provide a comprehensive audio representation of the environment. The signals from both the first microphone 702 and the second microphone 704 are fed into the ADC 706 to be digitized. This dual-microphone setup may be leveraged by the microcontroller unit 708 to perform signal processing tasks. For example, the microcontroller unit 708 may use beamforming techniques to focus on a sound source, such as a user speaking, while minimizing interference from other sounds in the room. In some implementations, the physical placement of the second microphone 704 relative to the first microphone 702 on the baby care device is configured to optimize audio quality. For example, the first microphone 702 and the second microphone 704 may be positioned on opposite sides of a device to capture a wide stereo field, which may be useful for localizing the source of a sound, such as identifying the direction from which a baby's cry is originating.

The ADC 706 may be configured to transform analog electrical signals from the first microphone 702 and the second microphone 704 into a digital audio stream. The ADC 706 may be an integrated circuit component within the baby care device. For example, the ADC 706 may sample the analog signals at a specific rate and bit depth to create a digital representation of the captured sound. The resulting digital audio stream may then be transmitted to the microcontroller unit 708 for processing. The ADC 706 may receive the analog outputs from the first microphone 702 and the second microphone 704 and perform a conversion process. The output of the ADC 706 is a digital data stream, which may be an interleaved stereo stream, that is then sent to the microcontroller unit 708, for instance, via an Inter-IC Sound (I2S) bus. The performance characteristics of the ADC 706, such as its sampling rate (e.g., 32,000 Hz) and resolution (e.g., 16-bit), may be selected to balance audio fidelity with the processing capabilities of the microcontroller unit 708. In some implementations, the ADC 706 may be part of a larger integrated circuit that includes other audio processing functionalities. For example, the ADC 706 may be integrated within a dedicated audio codec chip that also includes a digital-to-analog (DAC) converter. This integration may simplify the hardware design of the baby care device and reduce power consumption. The digital audio stream generated by the ADC 706 serves as the raw input for the on-device machine-learning pipeline.

The microcontroller unit 708 may be a processing component configured to execute instructions and perform computations for the baby care device. The microcontroller unit 708 may be, be similar to, include, or be included in the control device 110 shown in FIG. 1 or the processing circuitry 314 shown in FIG. 3. For example, the microcontroller unit 708 may be a low-power processor optimized for embedded systems, configured to receive the digital audio stream from the ADC 706 and perform on-device machine-learning inference. The microcontroller unit 708 may execute a series of data reduction and feature extraction operations on the audio stream received from the ADC 706. These operations may include down-sampling the audio and extracting acoustic features such as MFCCs. The microcontroller unit 708 may then provide these features to an embedded neural network model to identify a wakeword or user command. In the output path, the microcontroller unit 708 may generate audio signals to be sent to the codec 710 for playback through the speaker 714. In some implementations, the microcontroller unit 708 may be selected for its balance of computational power, memory capacity, and energy efficiency, making it suitable for a resource-constrained baby care device. For example, the microcontroller unit 708 may be an ESP32-S3, which includes processing capability to run a lightweight neural network model while consuming minimal power. The microcontroller unit 708 may store the machine-learning model and the processing pipeline software in its on-chip memory.

The codec 710 may be a coder-decoder component configured to perform digital-to-analog conversion. For example, the codec 710 may receive a digital audio signal from the microcontroller unit 708 and convert it into an analog electrical signal suitable for driving an amplifier. In some implementations, the codec 710 may be an integrated circuit that combines both analog-to-digital and digital-to-analog conversion functionalities, although in the audio processing pipeline 700 the codec 710 is shown in the output path. The codec 710 may receive digital audio data from the microcontroller unit 708, which may represent a synthesized voice response, an alert sound, or other audio. The codec 710 then processes this digital data and outputs a corresponding analog signal. This analog signal is then passed to the amplifier 712 to be strengthened before being sent to the speaker 714. In some implementations, the codec 710 may be part of an SoC that includes the microcontroller unit 708 and other peripheral components.

The amplifier 712 may be an electronic component configured to increase the power of an audio signal. The amplifier 712 may be an integrated circuit or a discrete component assembly. For example, the amplifier 712 receives the low-power analog audio signal from the codec 710 and boosts its amplitude to a level sufficient to drive the speaker 714 and produce audible sound. The amplifier 712 is a component in the audio output chain, positioned between the codec 710 and the speaker 714. The characteristics of the amplifier 712, such as its gain and power output, may be matched to the specifications of the speaker 714 to facilitate clear and audible sound reproduction. In some implementations, the amplifier 712 may include features such as volume control, which may be managed by the microcontroller unit 708. An amplifier, such as a Class-D amplifier, may be used to minimize power consumption during audio playback.

The speaker 714 may be an output transducer configured to convert an electrical audio signal into sound waves. The speaker 714 may be, be similar to, include, or be included in the speaker 320 shown in FIG. 3. For example, the speaker 714 may be used to provide audible feedback to a user, play sounds to an infant, or generate alerts. The speaker 714 receives the amplified analog signal from the amplifier 712 and physically vibrates to create sound that is audible. The size and type of the speaker 714 may be selected based on the design of the baby care device and the desired audio output quality. For example, a small speaker may be used in a wearable sensor, while a larger speaker may be included in a smart bassinet. In some implementations, the speaker 714 may be part of an integrated audio system that includes the amplifier 712 and other acoustic components designed to optimize sound quality.

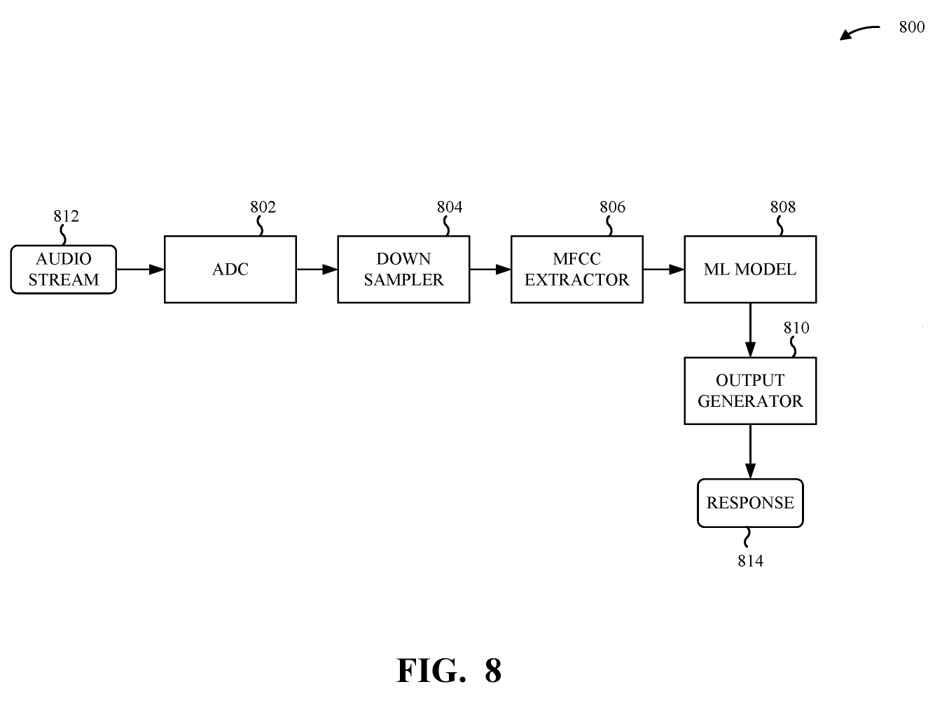

FIG. 8 is a block diagram of another example of an audio processing pipeline 800 of a baby care device. The audio processing pipeline 800 illustrates a sequence of software or processing operations for handling an audio stream 812 and generating a response 814 using a machine-learning model. The audio processing pipeline 800 may be implemented by a baby care device, such as the baby care device 102 shown in FIG. 1 or the baby care device 302 shown in FIG. 3. The audio processing pipeline 800 includes an ADC 802, a down sampler 804, an MFCC extractor 806, an ML model 808, an output generator 810, an audio stream 812, and a response 814.

The ADC 802 may be configured to receive an analog audio stream 812 and convert the analog audio stream 812 into a digital format. The audio stream 812 may be a data structure representing the sound captured from the environment. The audio stream 812 may be, be similar to, include, or be included in the audio stream 410 shown in FIG. 4. For example, the audio stream 812 may be an analog electrical signal generated by one or more microphones that captures user speech, infant vocalizations, or ambient noise. The ADC 802 may be, be similar to, include, or be included in the ADC 706 shown in FIG. 7. For example, the ADC 802 may receive an analog electrical signal from one or more microphones and digitize this signal to create a digital audio stream for processing. The output of the ADC 802 is a digital representation of the captured sound, which is then provided to the down sampler 804.

The down sampler 804 may be a component configured to reduce the sampling rate of the digital audio stream received from the ADC 802. For example, the down sampler 804 may generate a down-sampled digital audio stream based on down-sampling the digital audio stream from a first sample rate to a second, lower sample rate. This data reduction operation decreases the computational load on subsequent processing stages, which may be useful for resource-constrained devices. The output of the down sampler 804 is a lower-resolution digital audio stream that is provided to the MFCC extractor 806.

The MFCC extractor 806 may be a component configured to extract acoustic features from the down-sampled audio stream. For example, the MFCC extractor 806 may be configured to compute MFCCs. This feature extraction process may include segmenting the audio stream into frames, applying a windowing function, computing a Fast Fourier Transform (FFT), and applying a DCT. The extracted features are then provided to the ML model 808.

In some implementations, the process of extracting the set MFCCs may begin with a frame windowing step, where a windowing function, such as a 512-point Hanning window, is applied to each audio frame, which may have a duration of approximately 32 milliseconds. Following windowing, a Fast Fourier Transform (FFT) computation, such as a 512-point real-valued FFT, may be performed to convert the time-domain signal into the frequency domain, producing a set of magnitude bins. A power spectrum may then be computed, for example, by squaring the magnitude values and normalizing the result by the window power.

To align the frequency representation with human auditory perception, a Mel filter bank may be applied to the power spectrum. For example, a bank of 40 triangular filters spaced on the Mel scale may be used to project the power spectrum into a set of Mel bands. The Mel-scaled spectrum may then undergo log compression, for instance, by the application of a natural logarithm. A DCT may then be applied to the log-Mel spectrum to decorrelate the spectral bands and retain a compact set of coefficients. For example, the DCT may be used to convert the 40 Mel bands into the first 13 coefficients.

The resulting 13 coefficients may form an MFCC vector for the corresponding audio frame. These vectors, generated from a sequence of frames (e.g., 96 frames), may be assembled to form the feature tensor that is provided to the neural network model. Depending on the specific configuration of the model architecture, this feature tensor may have a shape such as [1, 13, 96] or [1, 16, 96]. In some implementations, alternative acoustic features may be generated. For example, a Mel Spectrogram, which may include 40 Mel-scaled spectral bins over 96 frames, may be used as the feature set without performing the final DCT step. In other implementations, to provide a richer representation of the audio, particularly in noisy conditions, delta and delta-delta features, which represent the temporal derivatives of the acoustic features, may be computed and included in the feature tensor.

The ML model 808 may be configured to perform an inference operation on the extracted acoustic features. The ML model 808 may be, be similar to, include, or be included in the ML model 318 shown in FIG. 3. For example, the ML model 808 may be a lightweight neural network, such as a DNN or an LSTM model, that is optimized for execution on an embedded processor. The ML model 808 may receive the features from the MFCC extractor 806 and produce an inference output, such as a probability score indicating the presence of a wakeword or a specific user command.

The output generator 810 may be a component configured to generate a response 814 based on the output of the ML model 808. For example, if the ML model 808 identifies a valid user command, the output generator 810 may formulate an appropriate action or audible reply. This may include generating a synthesized speech output, activating a device function, or preparing data for display. The output of the output generator 810 is a response 814, which may be provided to the user. For example, the response 814 may be an audible message played through a speaker, a visual indication on a display, or the execution of a specific device function, such as playing music. The response 814 is generated by the output generator 810 based on the inference performed by the ML model 808.

The sequence of operations in the audio processing pipeline 800 may facilitate efficient on-device processing by systematically reducing and transforming the audio stream 812 into a compact, feature-rich format suitable for a lightweight machine-learning model. The process may be initiated with the ADC 802 digitizing the incoming audio stream 812. The down sampler 804 may then reduce the computational burden by lowering the sample rate of the digital audio. Subsequently, the MFCC extractor 806 may convert the audio data into a set of acoustic features, which are a more informative and condensed representation of the sound. This feature set is then provided to the ML model 808, which may perform a rapid inference operation on the device's local hardware without the latency associated with cloud communication. The output generator 810 may translate the model's inference into a user-facing response 814, completing the process from audio capture to action entirely on the baby care device.

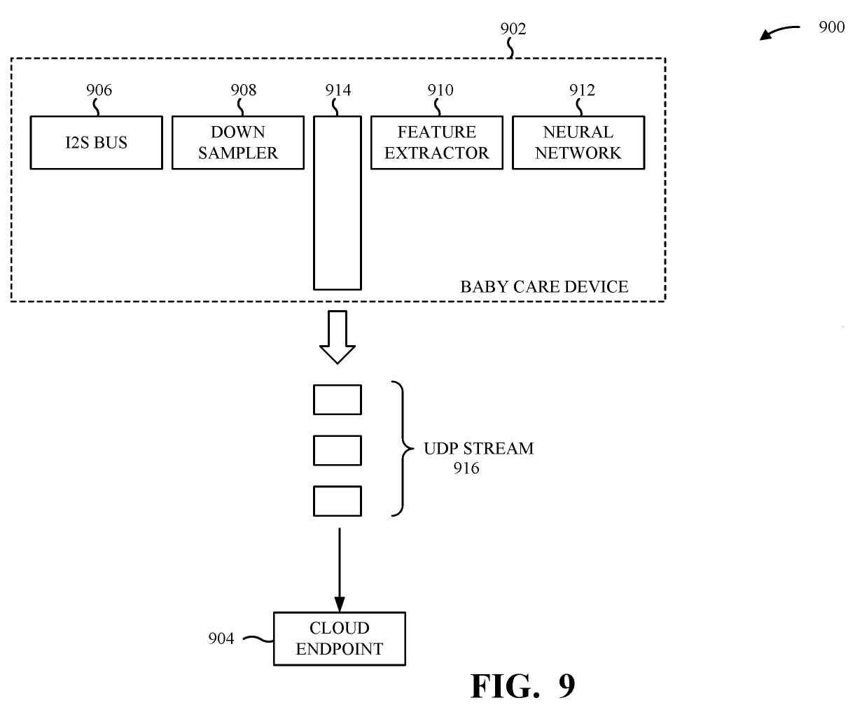

FIG. 9 is a conceptual block diagram of an example associated with a hybrid processing environment for an audio stream associated with baby care. The conceptual block diagram 900 illustrates a first implementation for a hybrid processing architecture where a baby care device 902 may transmit an audio stream to a cloud endpoint 904 for processing when an on-device model cannot fulfill a user command. In this implementation, the handoff to the cloud may occur after minimal on-device processing, with a down-sampled audio stream being transmitted.

The baby care device 902 may be, be similar to, include, or be included in the baby care device 102 shown in FIG. 1, the baby care device 302 shown in FIG. 3, or the baby care device 502 shown in FIG. 5. As shown in the conceptual block diagram 900, the baby care device 902 includes an on-device audio processing pipeline that may facilitate both local inference and the preparation of data for cloud offloading. The pipeline within the baby care device 902 may include an I2S bus 906, a down sampler 908, a feature extractor 910, a neural network 912, and a data buffer 914. The cloud endpoint 904 may be a network-accessible interface to a remote computing system, such as the cloud system 104 shown in FIG. 1 or the cloud system 304 shown in FIG. 3. The cloud endpoint 904 may be configured to receive and process data from the baby care device 902. In this implementation, the cloud endpoint 904 is configured to receive a raw audio stream and may perform functions such as speech-to-text conversion and natural language understanding using other cloud-based resources.

The I2S bus 906 may represent an initial stage of the on-device pipeline, providing a digital audio stream from one or more audio sensors. The I2S bus 906 may be, be similar to, include, or be included in the audio processing pipeline 700 shown in FIG. 7. The digital audio stream is then passed to the down sampler 908. The down sampler 908 may be a component or software module configured to reduce the sampling rate of the digital audio stream. The down sampler 908 may be, be similar to, include, or be included in the down sampler 804 shown in FIG. 8. This data reduction operation decreases the amount of data to be processed in subsequent stages, both on-device and for cloud transmission.

The feature extractor 910 may be a component configured to extract a set of acoustic features from the down-sampled audio stream. The feature extractor 910 may be, be similar to, include, or be included in the MFCC extractor 806 shown in FIG. 8. These features may be used by the neural network 912 for on-device inference. The neural network 912 may be an on-device machine-learning model configured to perform inference on the extracted acoustic features. The neural network 912 may be, be similar to, include, or be included in the ML model 318 shown in FIG. 3 or the ML model 808 shown in FIG. 8. The neural network 912 may identify a wakeword or a user command from the audio stream.

The data buffer 914 may be a component configured to access the audio processing pipeline at a specific point to prepare data for transmission to the cloud endpoint 904. In the conceptual block diagram 900, the data buffer 914 is positioned after the down sampler 908, indicating that the data buffer 914 captures the down-sampled, but otherwise unprocessed, digital audio stream. The data from the data buffer 914 is formatted into a UDP stream 916 for transmission.

The UDP stream 916 represents the data transmitted from the baby care device 902 to the cloud endpoint 904. In this architecture, the UDP stream 916 contains the raw, down-sampled audio data. Using a UDP stream may facilitate low-latency transmission, as UDP does not require the overhead of establishing a persistent connection or retransmitting lost packets, which may be suitable for real-time voice applications where some data loss is tolerable. This implementation may offload a greater amount of processing to the cloud, which may simplify the software complexity on the baby care device 902 for handling complex queries.

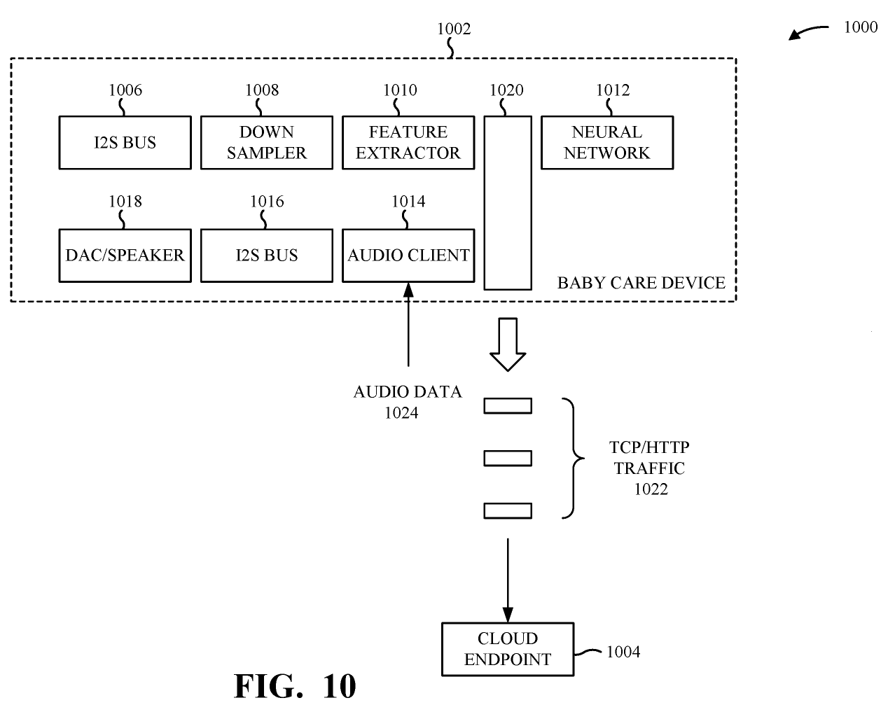

FIG. 10 is a conceptual block diagram of another example associated with a hybrid processing environment for an audio stream associated with baby care. The conceptual block diagram 1000 illustrates a second implementation for a hybrid processing architecture where a baby care device 1002 transmits extracted acoustic features to a cloud endpoint 1004. This approach differs from the one shown in FIG. 9 by performing more processing on-device to reduce the amount of data transmitted over a network.

The baby care device 1002 may be, be similar to, include, or be included in the baby care device 902 shown in FIG. 9. The baby care device 1002 includes an I2S bus 1006, a down sampler 1008, a feature extractor 1010, and a neural network 1012, which may be similar to their counterparts in FIG. 9. The baby care device 1002 further includes an audio client 1014, another I2S bus 1016, a DAC and speaker 1018, and a data buffer 1020. The cloud endpoint 1004 may be, be similar to, include, or be included in the cloud endpoint 904 shown in FIG. 9. In this architecture, the cloud endpoint 1004 is configured to receive audio data 1024, which contains acoustic features rather than raw audio, via TCP HTTP traffic 1022. The I2S bus 1006, down sampler 1008, feature extractor 1010, and neural network 1012 may perform functions analogous to the corresponding components described in FIG. 9. The audio pipeline processes an incoming audio stream for on-device inference.

The audio client 1014 may be a software component configured to manage the transmission of data to the cloud endpoint 1004. The audio client 1014 may be configured to format the audio data 1024 and communicate over a network protocol, such as TCP/HTTP. The I2S bus 1016 and the DAC and speaker 1018 represent components of an audio output path. After a response is received from the cloud endpoint 1004, the audio client 1014 may direct the audio data to a DAC and speaker for playback to the user. The data buffer 1020 may be a component that accesses the audio pipeline to capture data for cloud offloading. In this implementation, the data buffer 1020 is positioned after the feature extractor 1010, indicating that the data buffer 1020 captures the extracted acoustic features (e.g., MFCCs) rather than the raw audio stream. This captured data is then passed to the audio client 1014 for transmission.

The TCP HTTP traffic 1022 represents the communication between the audio client 1014 and the cloud endpoint 1004. Using TCP/HTTP may provide a reliable, connection-oriented data transfer, which may be suitable for structured acoustic feature data. The audio data 1024 represents the payload of this traffic, containing the set of acoustic features. This implementation may result in a reduction in network bandwidth compared to the architecture of FIG. 9, as the compact feature representation is smaller than the raw audio stream, which may be more efficient for devices with metered or slower network connections.

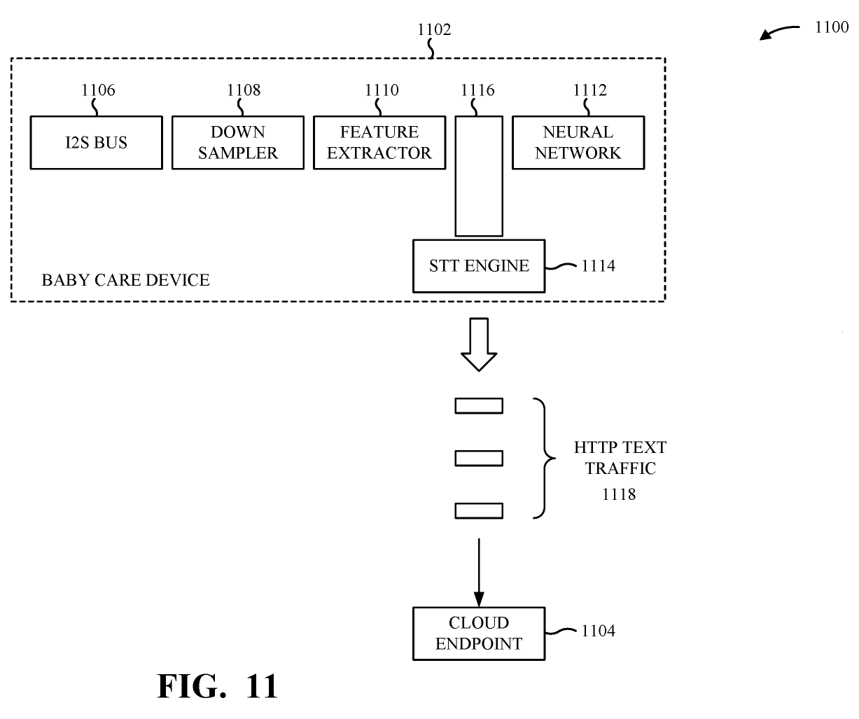

FIG. 11 is a conceptual block diagram of another example associated with a hybrid processing environment for an audio stream associated with baby care. The conceptual block diagram 1100 illustrates a third implementation for a hybrid processing architecture where a baby care device 1102 performs on-device speech-to-text conversion and transmits only text data to a cloud endpoint 1104. This architecture may maximize the amount of processing performed locally to achieve a high level of data efficiency and privacy.

The baby care device 1102 may be, be similar to, include, or be included in the baby care device 1002 shown in FIG. 10. The baby care device 1102 includes an I2S bus 1106, a down sampler 1108, a feature extractor 1110, and a neural network 1112, which may be similar to their counterparts in previous figures. The baby care device 1102 further includes an STT engine 1114 and a data buffer 1116. The cloud endpoint 1104 may be, be similar to, include, or be included in the cloud endpoint 1004 shown in FIG. 10. In this architecture, the cloud endpoint 1104 is configured to receive HTTP traffic 1118, which contains the textual representation of a user's query. The I2S bus 1106, down sampler 1108, feature extractor 1110, and neural network 1112 may perform functions analogous to the corresponding components described in previous figures, processing an audio stream for on-device analysis.

The STT engine 1114 may be a software component or dedicated hardware configured to convert acoustic features into a text string. The STT engine 1114 receives its input from the data buffer 1116, which accesses the pipeline after the feature extractor 1110. After converting the features to text, the STT engine 1114 provides the text string for transmission to the cloud endpoint 1104. The data buffer 1116 is positioned after the feature extractor 1110, capturing the acoustic features to be processed by the STT engine 1114. The HTTP traffic 1118 represents the communication between the baby care device 1102 and the cloud endpoint 1104. The payload of this traffic is plain text, which is a very compact data format. This implementation may require less network bandwidth and may offer greater data privacy, as a user's raw voice and its acoustic features are not transmitted from the baby care device 1102.

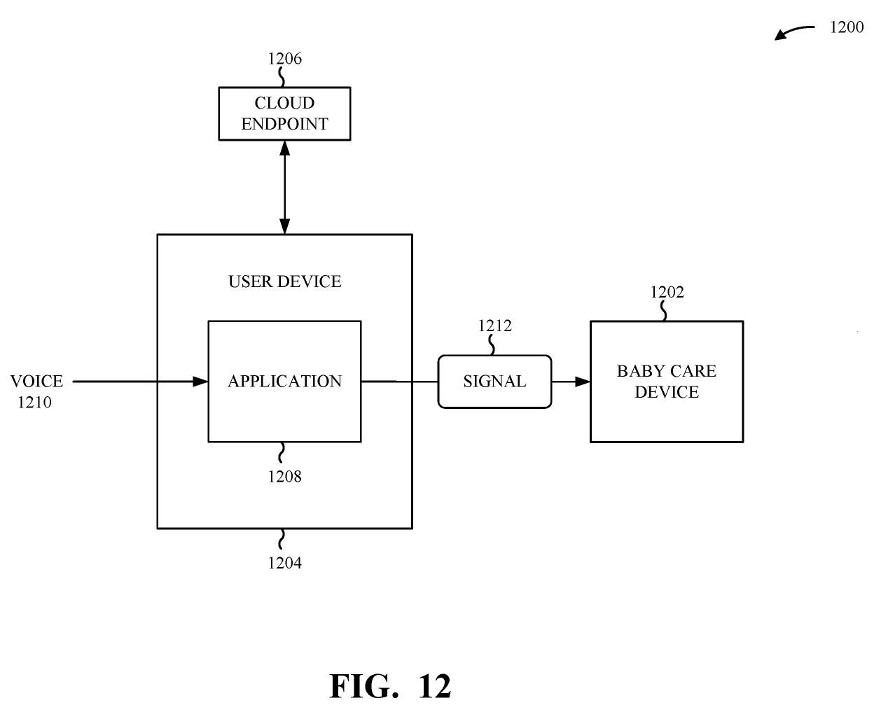

FIG. 12 is a conceptual block diagram of another example associated with a hybrid processing environment for an audio stream associated with baby care. The conceptual block diagram 1200 illustrates a fourth implementation for a hybrid processing architecture where the primary audio processing is offloaded from a baby care device 1202 to a separate user device 1204, such as a smartphone.

The baby care device 1202 may be, be similar to, include, or be included in the baby care device 1102 shown in FIG. 11, although in this architecture its role is simplified to that of a peripheral that receives commands. The user device 1204 may be a computing device, such as a smartphone, tablet, or personal computer, that runs a companion application 1208. The user device 1204 has its own processing, memory, and networking capabilities, which may be more powerful than those of the baby care device 1202. The cloud endpoint 1206 may be, be similar to, include, or be included in the cloud endpoint 1104 shown in FIG. 11. In this architecture, the cloud endpoint 1206 communicates with the application 1208 on the user device 1204, not directly with the baby care device 1202.

The application 1208 may be a software program running on the user device 1204. The application 1208 is configured to capture a user's voice 1210 using the microphone of the user device 1204. The application 1208 then handles the necessary processing, which may include speech-to-text conversion and communication with the cloud endpoint 1206 to resolve complex queries. The voice 1210 represents an audible utterance from a user, which is captured by the user device 1204 rather than the baby care device 1202.

The signal 1212 represents a command transmitted from the application 1208 on the user device 1204 to the baby care device 1202. This signal 1212 may be transmitted using a short-range wireless connection, such as Bluetooth Low Energy (BLE). The signal 1212 may be a simple command (e.g., "play music," "set temperature to 70 degrees") rather than a complex audio or feature stream. This implementation may offload complex audio and network processing from the resource-constrained baby care device 1202, which may simplify the hardware requirements, reduce the cost, and lower the power consumption of the baby care device 1202 itself.

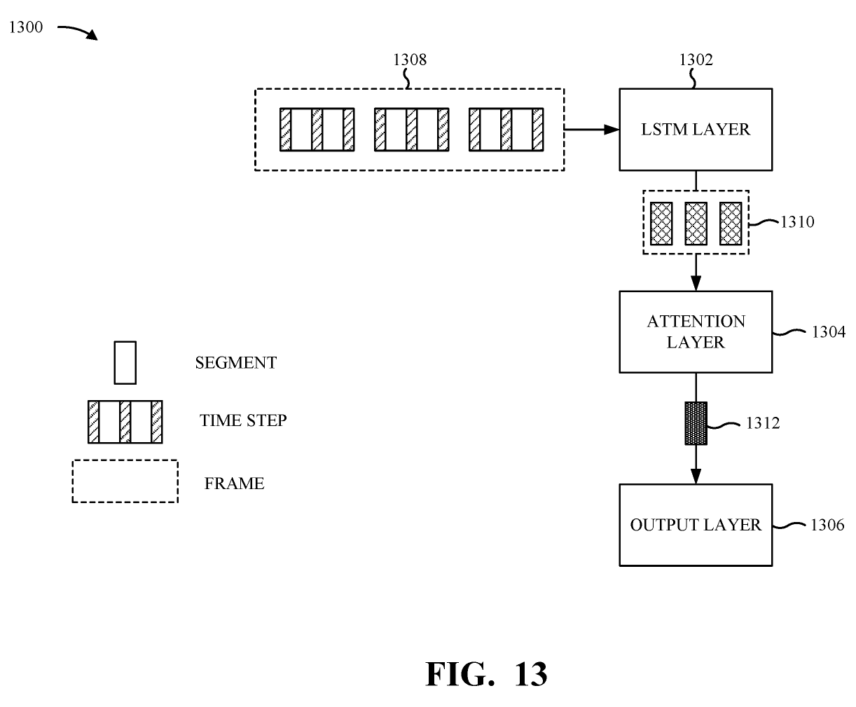

FIG. 13 is a block diagram of an example of a machine-learning model 1300 associated with processing an audio stream captured at a baby care device. The machine-learning model 1300 illustrates a neural network architecture, which may include an LSTM layer 1302, an attention layer 1304, and an output layer 1306. The machine-learning model 1300 may process an input frame 1308 to generate an inference output.

The machine-learning model 1300 may be, be similar to, include, or be included in the ML model 318 shown in FIG. 3 or the ML model 808 shown in FIG. 8. For example, the machine-learning model 1300 may be a lightweight neural network optimized for execution on a resource-constrained device, such as the baby care device 102 shown in FIG. 1. The architecture of the machine-learning model 1300 may be designed to efficiently process sequential data, such as a time series of acoustic features extracted from an audio stream.

The frame 1308 may be a data structure representing a segment of input data provided to the machine-learning model 1300. For example, the frame 1308 may be a feature tensor assembled from acoustic features, such as MFCCs, extracted from a series of overlapping audio frames. The frame 1308 may be structured as a sequence of time steps, where each time step corresponds to a set of features from a single audio frame.

The LSTM layer 1302 may be a component of the neural network configured to process sequential data. For example, the LSTM layer 1302 may be a type of recurrent neural network (RNN) layer that is capable of learning long-term dependencies in data by using a gating mechanism. The LSTM layer 1302 may receive the frame 1308 as input and process the frame 1308 sequentially, one time step at a time, to produce a sequence of hidden states 1310. The hidden states 1310 may be a data structure representing the output of the LSTM layer 1302. For example, the hidden states 1310 may be a sequence of vectors, where each vector encapsulates information from the current time step and all previous time steps in the input frame 1308. This sequence of hidden states 1310 is then provided as input to the attention layer 1304.

The attention layer 1304 may be a component of the neural network configured to weigh the importance of different parts of the input sequence. For example, the attention layer 1304 may be a mechanism that computes a set of attention weights for the sequence of hidden states 1310. These weights may indicate which time steps in the input sequence are most relevant for the current inference task. The attention layer 1304 may facilitate the generation of a context vector, which is a weighted sum of hidden states 1312. The weighted sum of hidden states 1312 may be a data structure representing a fixed-size context vector that summarizes relevant information from the entire input sequence. For example, the weighted sum of hidden states 1312 may be computed by multiplying each hidden state vector from the hidden states 1310 by its corresponding attention weight and summing the results. This context vector is then provided to the output layer 1306.

The output layer 1306 may be the final component of the neural network, configured to produce an inference output. For example, the output layer 1306 may be a fully connected layer followed by an activation function, such as a sigmoid function. The output layer 1306 receives the weighted sum of hidden states 1312 and generates a final output, which may be a probability score indicating the presence of a wakeword, a user command, or another target audio event.

The machine-learning model 1300 may represent one stage in a planned progression of models having incrementally greater complexity, all of which are engineered for efficient on-device deployment. For instance, a first inference operation may be performed by a first neural network having a first complexity, such as a simple DNN using Mel Spectrogram features for basic classification tasks. A subsequent, second inference operation may be performed by a second neural network having a second complexity that is greater than the first complexity, such as the machine-learning model 1300, which uses an LSTM layer to better process the sequential nature of speech data from MFCC features. This progression may continue to more advanced architectures, for example, a transformer model initially using an encoder for more sophisticated classification, followed by a full encoder-decoder transformer for sequence-generating tasks. Each stage in this evolution may be optimized to balance enhanced analytical capability with the operational constraints of the baby care device, such as limited power and memory, potentially using techniques such as post-training quantization to maintain efficiency. This architectural roadmap may facilitate the delivery of progressively more advanced AI functionalities to the device over time through software updates without altering the underlying hardware.

FIG. 14 is a flowchart of an example of a technique for on-device machine-learning processing for baby care devices. The technique 1400 may be executed using computing devices, such as the systems, hardware, and software described with respect to FIGS. 1-13. The technique 1400 may be performed, for example, by executing a machine-readable program or other computer-executable instructions, such as routines, instructions, programs, or other code. The steps, or operations, of the technique 1400, or another technique, method, process, or algorithm described in connection with the implementations disclosed herein may be implemented directly in hardware, firmware, software executed by hardware, circuitry, or a combination thereof. For simplicity of explanation, the technique 1400 is depicted and described herein as a series of steps or operations. However, the steps or operations of the technique 1400 may occur in various orders and/or concurrently. Additionally, other steps or operations not presented and described herein may be used. Furthermore, not all illustrated steps or operations may be required to implement a technique in accordance with the disclosed subject matter. The technique 1400 may be performed by a baby changing pad, such as the baby care device 102 shown in FIG. 1, configured to conduct on-device processing of user commands and provide a response to a user.

At 1402, the technique 1400 includes capturing an audio stream using at least one microphone of a baby changing pad. For example, the at least one microphone 114 of the baby care device 102 may be configured to capture an audio stream from the environment 100. The captured audio stream may include various sounds, such as the voice 116 of a user 106, infant vocalizations, or ambient background noise. The microphone 114 may convert these sound waves into an electrical signal, which is then digitized by an ADC, such as the ADC 706 shown in FIG. 7, to create the audio stream for on-device processing.

At 1404, the technique 1400 includes identifying, by one or more processors of the baby changing pad, a user command from the audio stream captured by the at least one microphone of the baby changing pad, wherein the user command is identified by extracting one or more acoustic features from the audio stream. For example, the processor set 204 of the baby changing pad may execute an audio processing pipeline, such as the audio processing pipeline 800 shown in FIG. 8. The processor set 204 may first generate a down-sampled audio stream and a single channel audio stream. The processor set 204 may then extract the one or more acoustic features, such as MFCCs, from the audio stream. The user command is then identified by processing the one or more acoustic features using at least one machine-learning model, such as the ML model 318, configured to run on the baby changing pad.

In some implementations, the technique 1400 may further include obtaining, using at least one physiological sensor of the baby changing pad, physiological measurements of a baby on the baby changing pad. One or more measurement features may be extracted from the physiological measurements. The one or more acoustic features and the one or more measurement features may be processed using the at least one machine-learning model to produce an inference, wherein the response is based on the inference. The at least one machine-learning model may include two or more machine-learning models, where each machine-learning model is associated with a corresponding patient risk of a set of patient risks including at least one of a physiological risk or a development risk.

At 1406, the technique 1400 includes generating, by the one or more processors, a response to the user command. For example, based on the inference output from the ML model 318, the output generator 810 may generate a response 814. The response may be formulated as an audible reply, a visual alert, or a command to control a function of the baby care device 302. In some implementations, the response indicates at least one of a patient risk score associated with a patient risk, an explanation of the patient risk score, or a care recommendation associated with the patient risk score.

At 1408, the technique 1400 includes outputting the response using at least one speaker of the baby changing pad. For example, the generated response 814 may be an audio signal that is sent through an audio output pipeline, such as the one depicted in FIG. 7, including a codec 710, an amplifier 712, and a speaker 714. The speaker 714 may then convert the electrical signal into audible sound, providing the response to the user 106. In some implementations, the response may be output via another output component, such as the display 112.

FIG. 15 is a flowchart of another example of a technique for on-device machine-learning processing for baby care devices. The technique 1500 may be executed using computing devices, such as the systems, hardware, and software described with respect to FIGS. 1-14. The technique 1500 may be performed, for example, by executing a machine-readable program or other computer-executable instructions, such as routines, instructions, programs, or other code. The steps, or operations, of the technique 1500, or another technique, method, process, or algorithm described in connection with the implementations disclosed herein may be implemented directly in hardware, firmware, software executed by hardware, circuitry, or a combination thereof. For simplicity of explanation, the technique 1500 is depicted and described herein as a series of steps or operations. However, the steps or operations of the technique 1500 may occur in various orders and/or concurrently. Additionally, other steps or operations not presented and described herein may be used. Furthermore, not all illustrated steps or operations may be required to implement a technique in accordance with the disclosed subject matter. The technique 1500 may be performed by a baby care device, such as the baby care device 302 shown in FIG. 3, configured to conduct on-device processing of baby physiological data to provide risk information to a user.

At 1502, the technique 1500 includes capturing an audio stream associated with a baby. For example, the at least one microphone 324 of the baby care device 302 may be configured to capture an audio stream from the environment. The audio stream may include infant vocalizations, respiratory sounds, or other acoustic data relevant to an infant's well-being. The microphone 324 may convert sound waves into an analog electrical signal.

At 1504, the technique 1500 includes receiving the audio stream. For example, the processing circuitry 314 of the baby care device 302 may receive a digital audio stream from an ADC, such as the ADC 706 shown in FIG. 7, which has digitized the analog signal from the microphone 324. The received digital audio stream may be the raw input for the on-device processing pipeline.

At 1506, the technique 1500 includes generating a down-sampled digital audio stream based on down-sampling the digital audio stream from a first sample rate to a second, lower sample rate. For example, the down sampler 804, executed by the processing circuitry 314, may reduce the sample rate of the received digital audio stream (e.g., from 32,000 Hz to 16,000 Hz). This data reduction operation may decrease the computational load for subsequent processing steps, which is an important consideration for a resource-constrained device.

At 1508, the technique 1500 includes generating the risk information by processing the down-sampled digital audio stream using at least one machine-learning model configured to run on the baby care device. For example, the processing circuitry 314 may execute an audio processing pipeline, such as the audio processing pipeline 800. In some implementations, this may include generating a single-channel audio stream, extracting a set of MFCCs, assembling the MFCCs into a feature tensor, and providing the feature tensor as input to the ML model 318. The ML model 318 may then produce an inference output that is used to generate the risk information. In some implementations, the risk information may indicate a patient risk score, an explanation of the score, or a care recommendation. In some implementations, the at least one machine-learning model includes at least one neural network.

At 1510, the technique 1500 includes outputting the risk information. For example, the baby care device 302 may use at least one output device, such as the speaker 320 or the display 322, to output the generated risk information to a user. An audible alert may be played through the speaker 320, or a visual notification with the risk score may be presented on the display 322.

Some implementations include a baby changing pad configured to conduct on-device processing of user commands and provide a response to a user, comprising: at least one microphone configured to capture an audio stream; at least one speaker configured to output sound; and one or more processors, individually or in combination, configured to: identify a user command from the audio stream captured by the at least one microphone of the baby changing pad, wherein the user command is identified by extracting one or more acoustic features from the audio stream; and generate a response to the user command that is output by the at least one speaker.

In some implementations, the baby changing pad comprises at least one physiological sensor configured to obtain physiological measurements of a baby on the baby changing pad, wherein the one or more processors, to generate the response, are further configured to: extract one or more measurement features from the physiological measurements; and process the one or more acoustic features and the one or more measurement features using at least one machine-learning model configured to run on the baby changing pad to produce an inference, wherein the response is based on the inference.

In some implementations, the at least one machine-learning model comprises: two or more machine-learning models, wherein each machine-learning model of the two or more machine-learning models is associated with a corresponding patient risk of a set of patient risks comprising at least one of a physiological risk or a development risk.

In some implementations, the response indicates at least one of a patient risk score associated with a patient risk, an explanation of the patient risk score, or a care recommendation associated with the patient risk score.

In some implementations, the one or more processors are configured to: transmit a set of data associated with a baby that has been placed on the baby changing pad to a cloud environment; and receive, from the cloud environment, a machine-learning model configured to run on the baby changing pad, wherein the machine-learning model is trained based on the set of data.

In some implementations, the machine-learning model comprises a neural network model.

In some implementations, the neural network model is one of a Long Short-Term Memory (LSTM) model, a transformer model, or a deep neural network (DNN) model.

In some implementations, the one or more processors are configured to perform an incremental inference operation by: processing a first chunk of the one or more acoustic features using an embedded neural network to generate a first output and an updated state; and processing a subsequent, second chunk of the one or more acoustic features using the embedded neural network and the updated state to generate a second output, wherein the response is based on at least one of the first output or the second output.

In some implementations, the one or more processors are configured to: generate a down-sampled audio stream by down-sampling the audio stream from a first sample rate to a second, lower sample rate; and generate a single channel audio stream based on the down-sampled audio stream.

In some implementations, to generate the response, the one or more processors are configured to: segment the audio stream into a set of overlapping audio frames using a sliding window implemented with a circular buffer; and perform an inference operation incrementally by processing one audio frame of the overlapping audio frames at a time.

In some implementations, to generate the response, the one or more processors are configured to: perform a first inference operation by providing the extracted one or more acoustic features to a first neural network running on the baby changing pad, the first neural network having a first complexity; and perform a second inference operation by providing the extracted one or more acoustic features to a second neural network running on the baby changing pad, the second neural network having a second complexity that is greater than the first complexity.

Some implementations include a method for conducting, by a baby changing pad, on-device processing of user commands and providing a response to a user, comprising: capturing an audio stream using at least one microphone of the baby changing pad; identifying, by one or more processors of the baby changing pad, a user command from the audio stream captured by the at least one microphone of the baby changing pad, wherein the user command is identified by extracting one or more acoustic features from the audio stream; generating, by the one or more processors, a response to the user command; and outputting the response using at least one speaker of the baby changing pad.

In some implementations, identifying the user command comprises: processing the one or more acoustic features using at least one machine-learning model configured to run on the baby changing pad.

In some implementations, the method further comprises: obtaining, using at least one physiological sensor of the baby changing pad, physiological measurements of a baby on the baby changing pad; extracting one or more measurement features from the physiological measurements; and processing the one or more acoustic features and the one or more measurement features using at least one machine-learning model configured to run on the baby changing pad to produce an inference, wherein the response is based on the inference.

In some implementations, the method further comprises: transmitting a set of data associated with a baby that has been placed on the baby changing pad to a cloud environment; and receiving, from the cloud environment, a machine-learning model configured to run on the baby changing pad, wherein the machine-learning model is trained based on the set of data.

In some implementations, the method further comprises: segmenting the audio stream into a set of overlapping audio frames using a sliding window; and performing an incremental inference operation by incrementally processing the set of overlapping audio frames to generate an inference output, wherein the response is based on the inference output.

Some implementations include a baby care device configured to conduct on-device processing of baby physiological data to provide risk information to a user, comprising: at least one microphone configured to capture an audio stream associated with a baby; at least one output device configured to output risk information; and one or more processors, individually or in combination, configured to: receive the audio stream; generate a down-sampled digital audio stream based on down-sampling the digital audio stream from a first sample rate to a second, lower sample rate; and generate the risk information by processing the down-sampled digital audio stream using at least one machine-learning model configured to run on the baby care device.

In some implementations, the one or more processors, to process the down-sampled digital audio stream, are configured to: generate a single-channel audio stream based on the down-sampled digital audio stream; extract a set of Mel Frequency Cepstrum Coefficients (MFCCs) from the single-channel audio stream; assemble the set of MFCCs into a feature tensor; and provide the feature tensor as an input to the at least one machine-learning model to generate an inference output, wherein the risk information is based on the inference output.

In some implementations, to extract the set of MFCCs, the one or more processors are configured to: perform an initialization operation associated with the single-channel audio stream; generate a set of frame segments by performing a frame segmentation operation associated with the single-channel audio stream; determine a power spectrum associated with the set of frame segments; and determine the set of MFCCs based on computing a discrete cosine transform (DCT) of the power spectrum.

In some implementations, the at least one machine-learning model comprises at least one neural network.

The implementations of this disclosure can be described in terms of functional block components and various processing operations. Such functional block components can be realized by a number of hardware or software components that perform the specified functions. For example, the disclosed implementations can employ various integrated circuit components (e.g., memory elements, processing elements, logic elements, look-up tables, and the like), which can carry out a variety of functions under the control of one or more microprocessors or other control devices. Similarly, where the elements of the disclosed implementations are implemented using software programming or software elements, the systems and techniques can be implemented with a programming or scripting language, such as C, C++, Java, JavaScript, assembler, or the like, with the various algorithms being implemented with a combination of data structures, objects, processes, routines, or other programming elements.

Functional aspects can be implemented in algorithms that execute on one or more processors. Furthermore, the implementations of the systems and techniques disclosed herein could employ a number of conventional techniques for electronics configuration, signal processing or control, data processing, and the like. The words “mechanism” and “component” are used broadly and are not limited to mechanical or physical implementations, but can include software routines in conjunction with processors, etc. Likewise, the terms “system” or “tool” as used herein and in the figures, but in any event based on their context, may be understood as corresponding to a functional unit implemented using software, hardware (e.g., an integrated circuit, such as an ASIC), or a combination of software and hardware. In certain contexts, such systems or mechanisms may be understood to be a processor-implemented software system or processor-implemented software mechanism that is part of or callable by an executable program, which may itself be wholly or partly composed of such linked systems or mechanisms.

Implementations or portions of implementations of the above disclosure can take the form of a computer program product accessible from, for example, a computer-usable or computer-readable medium. A computer-usable or computer-readable medium can be a device that can, for example, tangibly contain, store, communicate, or transport a program or data structure for use by or in connection with a processor. The medium can be, for example, an electronic, magnetic, optical, electromagnetic, or semiconductor device.

Other suitable mediums are also available. Such computer-usable or computer-readable media can be referred to as non-transitory memory or media, and can include volatile memory or non-volatile memory that can change over time. The quality of memory or media being non-transitory refers to such memory or media storing data for some period of time or otherwise based on device power or a device power cycle. A memory of an apparatus described herein, unless otherwise specified, does not have to be physically contained by the apparatus, but is one that can be accessed remotely by the apparatus, and does not have to be contiguous with other memory that might be physically contained by the apparatus.

As used herein, satisfying a threshold may, depending on the context, refer to a value being greater than the threshold, greater than or equal to the threshold, less than the threshold, less than or equal to the threshold, equal to the threshold, not equal to the threshold, or the like.

Even though particular combinations of features are recited in the claims and/or disclosed in the specification, these combinations are not intended to limit the disclosure of various aspects. In fact, many of these features may be combined in ways not specifically recited in the claims and/or disclosed in the specification. Although each dependent claim listed below may directly depend on only one claim, the disclosure of various aspects includes each dependent claim in combination with every other claim in the claim set. As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the terms “set” and “group” are intended to include one or more items (e.g., related items, unrelated items, or a combination of related and unrelated items), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

The adjectives “first,” “second,” “third,” and so on are used for contextual distinction between two or more of the modified nouns in connection with a discussion and are not meant to be absolute modifiers that apply only to a certain respective node throughout the entire document. For example, a component may be referred to as a “first component” in connection with one discussion and may be referred to as a “second component” in connection with another discussion, or vice versa. Reference to a component, a computing device, a server, a client, an application, an apparatus, a device, a system, a computing system, or the like may include disclosure of the computing device, server, client, application, apparatus, device, system, computing system, or the like, respectively, being a node. For example, disclosure that a computing device is configured to receive information from a server also discloses that a first node is configured to receive information from a second node. Consistent with this disclosure, once a specific example is broadened in accordance with this disclosure (e.g., a computing device is configured to receive information from a server also discloses that a first node is configured to receive information from a second node), the broader example of the narrower example may be interpreted in the reverse, but in a broad open-ended way. In the example above where a computing device being configured to receive information from a server also discloses a first node being configured to receive information from a second node, “first node” may refer to a first computing device, a first server, a first client, a first application, a first apparatus, a first device, a first system, a first computing system, or the like, configured to receive the information from a second node; and “second node” may refer to a second computing device, a second server, a second client, a second application, a second apparatus, a second device, a second system, a second computing system, or the like.

While the disclosure has been described in connection with certain implementations, it is to be understood that the disclosure is not to be limited to the disclosed implementations but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A baby changing pad configured to conduct on-device processing of user commands and provide a response to a user, comprising:
at least one microphone configured to capture an audio stream;
at least one speaker configured to output sound; and
one or more processors, individually or in combination, configured to:
identify a user command from the audio stream captured by the at least one microphone of the baby changing pad, wherein the user command is identified by extracting one or more acoustic features from the audio stream; and
generate a response to the user command that is output by the at least one speaker.

2. The baby changing pad of claim 1, comprising at least one physiological sensor configured to obtain physiological measurements of a baby on the baby changing pad, wherein the one or more processors, to generate the response, are further configured to:
extract one or more measurement features from the physiological measurements; and
process the one or more acoustic features and the one or more measurement features using at least one machine-learning model configured to run on the baby changing pad to produce an inference, wherein the response is based on the inference.

3. The baby changing pad of claim 2, wherein the at least one machine-learning model comprises:
two or more machine-learning models, wherein each machine-learning model of the two or more machine-learning models is associated with a corresponding patient risk of a set of patient risks comprising at least one of a physiological risk or a development risk.

4. The baby changing pad of claim 1, wherein the response indicates at least one of a patient risk score associated with a patient risk, an explanation of the patient risk score, or a care recommendation associated with the patient risk score.

5. The baby changing pad of claim 1, wherein the one or more processors are configured to:
transmit a set of data associated with a baby that has been placed on the baby changing pad to a cloud environment; and
receive, from the cloud environment, a machine-learning model configured to run on the baby changing pad, wherein the machine-learning model is trained based on the set of data.

6. The baby changing pad of claim 5, wherein the machine-learning model comprises a neural network model.

7. The baby changing pad of claim 6, wherein the neural network model is one of a Long Short-Term Memory (LSTM) model, a transformer model, or a deep neural network (DNN) model.

8. The baby changing pad of claim 1, wherein the one or more processors are configured to perform an incremental inference operation by:
processing a first chunk of the one or more acoustic features using an embedded neural network to generate a first output and an updated state; and
processing a subsequent, second chunk of the one or more acoustic features using the embedded neural network and the updated state to generate a second output, wherein the response is based on at least one of the first output or the second output.

9. The baby changing pad of claim 1, wherein the one or more processors are configured to:
generate a down-sampled audio stream by down-sampling the audio stream from a first sample rate to a second, lower sample rate; and
generate a single channel audio stream based on the down-sampled audio stream.

10. The baby changing pad of claim 1, wherein, to generate the response, the one or more processors are configured to:
segment the audio stream into a set of overlapping audio frames using a sliding window implemented with a circular buffer; and
perform an inference operation incrementally by processing one audio frame of the overlapping audio frames at a time.

11. The baby changing pad of claim 1, wherein, to generate the response, the one or more processors are configured to:
perform a first inference operation by providing the extracted one or more acoustic features to a first neural network running on the baby changing pad, the first neural network having a first complexity; and
perform a second inference operation by providing the extracted one or more acoustic features to a second neural network running on the baby changing pad, the second neural network having a second complexity that is greater than the first complexity.

12. A method for conducting, by a baby changing pad, on-device processing of user commands and providing a response to a user, comprising:
capturing an audio stream using at least one microphone of the baby changing pad;
identifying, by one or more processors of the baby changing pad, a user command from the audio stream captured by the at least one microphone of the baby changing pad, wherein the user command is identified by extracting one or more acoustic features from the audio stream;
generating, by the one or more processors, a response to the user command; and
outputting the response using at least one speaker of the baby changing pad.

13. The method of claim 12, wherein identifying the user command comprises:
processing the one or more acoustic features using at least one machine-learning model configured to run on the baby changing pad.

14. The method of claim 12, further comprising:
obtaining, using at least one physiological sensor of the baby changing pad, physiological measurements of a baby on the baby changing pad;
extracting one or more measurement features from the physiological measurements; and
processing the one or more acoustic features and the one or more measurement features using at least one machine-learning model configured to run on the baby changing pad to produce an inference, wherein the response is based on the inference.

15. The method of claim 12, further comprising:
transmitting a set of data associated with a baby that has been placed on the baby changing pad to a cloud environment; and
receiving, from the cloud environment, a machine-learning model configured to run on the baby changing pad, wherein the machine-learning model is trained based on the set of data.

16. The method of claim 15, further comprising:
segmenting the audio stream into a set of overlapping audio frames using a sliding window; and
performing an incremental inference operation by incrementally processing the set of overlapping audio frames to generate an inference output, wherein the response is based on the inference output.

17. A baby care device configured to conduct on-device processing of baby physiological data to provide risk information to a user, comprising:
at least one microphone configured to capture an audio stream associated with a baby;
at least one output device configured to output risk information; and
one or more processors, individually or in combination, configured to:
receive the audio stream;
generate a down-sampled digital audio stream based on down-sampling the digital audio stream from a first sample rate to a second, lower sample rate; and
generate the risk information by processing the down-sampled digital audio stream using at least one machine-learning model configured to run on the baby care device.

18. The baby care device of claim 17, wherein the one or more processors, to process the down-sampled digital audio stream, are configured to:
generate a single-channel audio stream based on the down-sampled digital audio stream;
extract a set of Mel Frequency Cepstrum Coefficients (MFCCs) from the single-channel audio stream;
assemble the set of MFCCs into a feature tensor; and
provide the feature tensor as an input to the at least one machine-learning model to generate an inference output, wherein the risk information is based on the inference output.

19. The baby care device of claim 18, wherein, to extract the set of MFCCs, the one or more processors are configured to:
perform an initialization operation associated with the single-channel audio stream;
generate a set of frame segments by performing a frame segmentation operation associated with the single-channel audio stream;
determine a power spectrum associated with the set of frame segments; and
determine the set of MFCCs based on computing a discrete cosine transform (DCT) of the power spectrum.

20. The baby care device of claim 17, wherein the at least one machine-learning model comprises at least one neural network.
